(19) United States
(12) Patent Application Publication
(10) Pub. No.: US 2019/0008795 A1
(43) Pub. Date: Jan. 10, 2019

(54) COMPOSITIONS FOR TOPICAL APPLICATION OF COMPOUNDS

(71) Applicant: Illustris Pharmaceuticals, Inc., Palo Alto, CA (US)

(72) Inventor: Jacob M. WAUGH, Corona del Mar, CA (US)

(21) Appl. No.: 16/131,859

(22) Filed: Sep. 14, 2018

Related U.S. Application Data
(62) Division of application No. 15/486,240, filed on Apr. 12, 2017.
(60) Provisional application No. 62/321,626, filed on Apr. 12, 2016.

(51) Int. Cl. A61K 9/70; A61K 8/02; A61Q 19/06; A61Q 17/04; A61K 8/73; A61Q 7/00; A61K 8/65; A61K 8/64; A61K 8/42; A61K 31/573; A61K 47/42; A61K 47/36; A61K 9/00

(52) U.S. Cl. CPC A61K 9/7023; A61K 2800/92; A61K 38/4893; A61K 2800/56; A61K 8/0241; A61Q 19/06; A61Q 17/04; A61K 8/735; A61Q 7/00; A61K 8/65; A61K 8/64; A61K 8/42; A61K 31/573; A61K 47/42; A61K 47/36; A61K 9/0014

(57) ABSTRACT

Compositions for transdermal delivery of an active agent and methods for using such compositions are described herein.

### CROSS-REFERENCE TO RELATED APPLICATIONS

This application is a divisional of U.S. application Ser. No. 15/486,240 filed on Apr. 12, 2017, which claims the benefit of U.S. Provisional Application No. 62/321,626, entitled “Compositions for Topical Application of Compounds,” filed Apr. 12, 2016, and are incorporated herein by reference in their entirety.

### BACKGROUND

A topical route of drug administration is desirable because the risks and inconvenience of parenteral treatment can be avoided; the variable absorption and metabolism associated with oral treatment can be circumvented; drug administration can be continuous, thereby permitting the use of pharmacologically active agents with short biological half-lives; the gastrointestinal irritation associated with many compounds can be avoided; and cutaneous manifestations of diseases can be treated more effectively than by systemic approaches. Most transdermal and transmucosal delivery systems achieve penetration by using a penetration-enhancing vehicle. Such compounds or mixtures of compounds are known in the art as “penetration enhancers” or “skin enhancers.” Many of the penetration enhancers in the literature enhance transdermal absorption, yet they also possess certain drawbacks in that some are regarded as toxic; some irritate the skin; some have a thinning effect on the skin on prolonged use; and most are incapable of delivering high molecular weight pharmaceuticals and cosmetic agents. Clearly, there remains a need for safe and effective transdermal delivery compositions and systems that can administer a wide-range of pharmaceuticals and cosmetic agents to and through the skin, mucosa, hair, nails, teeth, and various other surfaces.

### BRIEF SUMMARY

Various embodiments of the invention include compositions containing one or more active agents and about 0.1 wt. % to about 5.0 wt. % of a extracellular matrix component or a fragment thereof having average molecular weight of about 2,000 daltons to about 60,000 daltons. In some embodiments, the decoy molecule may be selected from the group consisting of hyaluronic acid, collagen, fibronectin, elastin, lectin, and combinations thereof, and in certain embodiments, the collagen may be selected from the group consisting of collagen type I, collagen type II, collagen type III, collagen type IV, collagen type V, fibrillary collagen, non-fibrillary collagen, and combinations thereof

In particular embodiments, the compositions may include about 1 mg to about 1000 mg of the extracellular matrix component or a fragment thereof In some embodiments, the compositions may include about 0.1 wt. % to about 25 wt. % active agent, and in some embodiments, the compositions may include about 1 mg to about 1000 mg active agent. In various embodiments, the active agent may be selected from the group consisting of analgesic agents, antibacterial agents, antifungal agents, anesthetics, steroids, retinol, gabapentin, pregabalin, minocycline, salicylate, acetyl salicylic acid, cyclosporine, tacrolimus (FK506), hydrocortisone, lidocaine, bimatoprost, botulinum toxin, tadalafil, an antibody, an antibody fragment, and the like or combinations thereof.

The compositions of embodiments may be formulated as a liquid, cream, ointment, gel, or aerosol. In some embodiments, the compositions my further include one or more pharmaceutical additives selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, or combinations thereof. In some embodiments, the compositions may further include one or more cosmetic additives selected from the group consisting of vitamins, cosmetic peptides, oil control agents, other skin care agents, and hydrating compositions. In some embodiments, the composition may further include a compound that absorbs or reflects UV photons.

In particular embodiments, the compositions may include about 0.25 wt. % to about 2.0 wt. % of the decoy molecule wherein the active agent is selected from the group consisting of salicylate, lidocaine, sunblock, retinol, bimatoprost, steroids, and combinations thereof. In certain embodiments, the compositions may include about 1.0 wt. % to about 5.0 wt. % of the decoy molecule wherein the active agent is selected from the group consisting of antibiotics, antifungal agents, biologics, antibodies, macromolecule active agents, peptide-based therapeutics, and combinations thereof.

Further embodiments include methods for delivering an active agent including the steps of applying to a surface tissue of a subject a composition comprising one or more active agents and about 0.25 wt. % to about 10 wt. % of a extracellular matrix component or a fragment thereof having average molecular weight of about 2,000 daltons to about 60,000 daltons. In particular embodiments, the decoy molecule may be selected from the group consisting of hyaluronic acid, collagen, fibronectin, elastin, lectin, and fragments and combinations thereof.

In particular embodiments, the compositions of such methods may include about 1 mg to about 1000 mg of the extracellular matrix component or a fragment thereof. In some embodiments, the compositions of such methods may include about 0.1 wt. % to about 25 wt. % active agent, and in some embodiments, the compositions of such methods may include about 1 mg to about 1000 mg active agent. In various embodiments, the active agent may be selected from the group consisting of analgesic agents, antibacterial agents, antifungal agents, anesthetics, steroids, retinol, gab apentin, pregabalin, minocycline, salicylate, acetyl salicylic acid, cyclosporine, tacrolimus (FK506), hydrocortisone, lidocaine, bimatoprost, botulinum toxin, tadalafil, an antibody, an antibody fragment, and the like or combinations thereof.

### BRIEF DESCRIPTION OF THE DRAWINGS

For a fuller understanding of the nature and advantages of the present invention, reference should be made to the following detailed description taken in connection with the accompanying drawings, in which:

FIGS. 1A-1B are graphs showing the percent of peptide flux relative to flux of peptide from the composition of peptide alone, for peptide compositions comprising a decoy molecule of hyaluronic acid with a molecular weight of 10,000 Da, 20,000 Da, 40,000 Da, 60,000 Da, or 100,000 Da, where flux was measured in skin with stratum corneum intact (FIG. 1A) and in skin with stratum corneum stripped (FIG. 1B) and each composition was measured in duplicate (solid line, dashed line).

FIG. 2 is a bar graph showing the percent increase of salicylate flux from compositions of salicylate and a decoy molecule of hyaluronic acid with molecular weights designated as small (5,000 Da to 10,000 Da), small to mid (10,000 Da to 20,000 Da), low to mid (20,000 Da to 30,000 Da), and mid (30,000 Da to 40,000 Da) over a composition with no decoy molecule.

FIG. 3 is a bar graph showing the percent increase of hydrocortisone flux from compositions of hydrocortisone and a decoy molecule of hyaluronic acid with molecular weights designated as very small (5,000 Da to 10,000 Da), small (10,000 Da to 20,000 Da), mid (30,000 Da to 40,000 Da), and large (40,000 Da to 60,000 Da) over a composition with no decoy molecule.

FIG. 4 is a bar graph showing the percent of lidocaine in porcine skin from topically applied compositions of lidocaine and an elastin decoy molecule with a molecular weight designated as very very small (2,000 Da to 5,000 Da), very small (5,000 Da to 10,000 Da), and small (10,000 Da to 20,000 Da) and no decoy molecule.

FIG. 5 is a bar graph showing the percent of topically applied minocycline in porcine skin from compositions containing of minocycline and a decoy molecule of hyaluronic acid with molecular weights designated as 3,000 Da, 5,000 Da, and 10,000 Da compared with a composition with no decoy.

FIG. 6 is a bar graph showing the absorption of UVA and UVB in skin (4.0 corresponds to 100%), where the bars correspond with a sunscreen composition with a decoy molecule added to the commercially available sunscreen (Anthelios 60) and the commercially available sunscreen (Anthelios 60).

FIGS. 7A-7B are graphs of UV absorption as a function of wavelength, in nm, for commercially available sunscreen (Anthelios 60) alone (FIG. 7A) and for the commercially available sunscreen (Anthelios 60) with a decoy molecule (FIG. 7B).

FIG. 8 is a graph showing the percent UV absorbance through skin as a function of wavelength, in nm, for commercially available sunscreen (Anthelios 60) (solid line) and for the commercially available sunscreen (Anthelios 60) with a decoy molecule (dashed line).

FIG. 9 is a bar graph showing the amount of gabapentin in tissue (μg gabapentin/g tissue) delivered into porcine skin grafts in vitro from a topical formulation of gabapentin and sodium hyaluronate and from a topical formulation of gabapentin alone.

FIG. 10 is a bar graph showing the amount of palmitoyl-lysine-threonine-threonine-lysine-serine (pal-KTTKS) in tissue (μg pal-KTTKS/50 mg tissue) delivered into porcine skin grafts in vitro from a topical formulation of pal-KTTKS and sodium hyaluronate and from a topical formulation of pal-KTTKS alone.

FIG. 11 is a bar graph showing the percent increase in salicylate delivery across porcine mucosal tissue when a decoy molecule of elastin is included in the composition compared with a composition of salicylate and saline.

FIG. 12 is bar graph showing the percentage increase of antibody flux from compositions comprised of antibody and a decoy molecule of hyaluronic acid with molecular weights designated as vvlow, vlow and low compared with antibody alone.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The compositions of the present disclosure can comprise, consist essentially of, or consist of, the components disclosed.

All percentages, parts and ratios are based upon the total weight of the topical compositions and all measurements made are at about 25° C., unless otherwise specified.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subj ect.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition in a subject relative to a subject not receiving the compound or composition. This can include reversing, reducing, or arresting the symptoms, clinical signs, and underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments of the invention are directed to compositions containing an active agent and a decoy molecule that is capable of causing rearrangement of tissues that the composition contacts by temporarily disrupting cell-cell (i.e. intercellular) and cell-scaffold attachment allowing the active agent to pass through cell layers and passive intracellular crossing of the active agent into cells throughout the tissue. Further embodiments include methods for administering an active agent by contacting a tissue with a composition containing an active agent and a decoy molecule. The compositions and methods described herein can be used for administering any active agent including small molecule drugs, macromolecular drugs, biologics, antibodies, chimeric antibodies, peptides, antioxidants, and the like and combinations thereof. The compositions and methods can also be used for diagnostic purposes and mediating the flow of diagnostic molecules through various tissues. The compositions can be applied to any surface tissue, including skin, mucosa, eyes, ears, inside the nose, inside the mouth, lips, urethral openings, vaginal, anus, tongue, frenulum of tongue, hair, teeth, and the like.

In certain embodiments, the decoy molecule may be an extracellular matrix component or a fragment thereof or a receptor associated with the extracellular matrix. For example, in some embodiments, the decoy molecule may be hyaluronic acid, elastin, collagen, fibronectin, lectin, and the like and combinations thereof

The size of the decoy molecule may impact the cell-cell and cell-scaffold disruption, and in various embodiments, the decoy molecule may have an average molecular weight of less than 100,000 Daltons (“Da”). In particular embodiments, the decoy molecule may have an average molecular weight of about 2,000 Da to about 60,000, about 2,000 Da to about 40,000 Da, or about 5,000 Da to about 40,000 Da. In other embodiments, the decoy molecule may have an average molecular weight of about 2,000 Da to about 5,000 Da (“very small” size), about 5,000 Da to about 10,000 Da (“small” size), about 10,000 Da to about 20,000 Da (“small-to-mid” size), about 20,000 Da to about 30,000 Da (“low-to-mid” size), about 30,000 Da to about 40,000 Da (“mid” size), about 40,000 Da to about 60,000 Da (“large” size), or about 60,000 Da to about 100,000 Da (“very large” size). Because the decoy molecule generally includes fragments of extracellular matrix components, the compositions of embodiments may include decoy molecules falling within any of the ranges identified above and outside the “average molecular weight.” For example, the decoy molecule may include individual molecules that are large and extra-large or very small and small when the average molecular weight is small-to-mid.

The amount of decoy in the composition may impact the cell-cell and cell-scaffold disruption by modulating the depth of the disruption, thereby modulating the depth of penetration of the active. In general, the amount of decoy present in the compositions of various embodiments may be from about 0.1 wt. % to about 10 wt. %, and in particular embodiments, the amount of decoy in such compositions may be from about 0.1 wt. % to about 2.0 wt. %, about 0.25 wt. % to about 3.0 wt. %, about 0.5 wt. % to about 5.0 wt. %, about 0.75 wt. % to about 7.5 wt. %, or any range or individual concentration encompassing these example ranges. As indicated above, the amount of decoy molecule can modulate the depth of penetration of the active agent. For example, a relatively low concentration of decoy molecule, e.g. about 0.1 wt. % to about 2.0 wt. % or about 0.25 wt. % to about 1.0 wt. %, may allow for transport of an active agent partially across the epidermis, for example, through the stratum granulosum and into the stratum spinosum, when the composition is administered topically. A higher concentration of decoy molecule, e.g. about 0.5 wt. % to about 5.0 wt. % or about 0.5 wt. % to about 3.0 wt. %, may allow for transport of an active agent fully across the epidermis to the basement membrane underlying tissues layers, for example, dermis, subcutis, and blood stream, when the composition is administered topically. In some embodiments, the amount of decoy molecule in a composition may be about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 mg, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values.

Because the concentration of decoy molecule can modulate the depth of penetration of the active agent, active agents that target, for example, the epidermis may be included in compositions containing lower concentrations of decoy molecule, e.g. about 0.1 wt. % to about 2.0 wt. % or about 0.25 wt. % to about 1.5 wt. %, and active agents that target, for example, dermis or subcutanis may be included in compositions containing higher concentrations of decoy molecule, e.g. about 1.0 wt. % to about 5.0 wt. % or about 1.0 wt. % to about 3.0 wt. %. Similarly, the size of the active agent may impact the formulation of the composition. For example, a large active agent, such as a macromolecule therapeutic or biologic/therapeutic peptide may require higher concentrations of decoy molecule, e.g. about 0.5 wt. % to about 5.0 wt. % or about 0.5 wt. % to about 3.0 wt. %, to allow administration to the epidermis even though similar concentrations may allow administration of smaller therapeutics to the dermis or systemic administration through the blood stream.

In particular embodiments, the decoy molecule may be hyaluronic acid. Hyaluronic acid is known to interact with, for example, CD44, receptor for hyaluronic acid-mediated motility (RHAMM), and intercellular adhesion molecule-1 (ICAM-1). CD44 is widely distributed throughout the body and mediates cell interaction with hyaluronic acid. ICAM-1 is a metabolic cell surface receptor for hyaluronic acid, and binding of hyaluronic acid to ICAM-1 may contribute to the control of ICAM-1-mediated inflammatory activation. Hyaluronic acid is polymer of disaccharides. Without wishing to be bound by theory, low molecular weight fragments of hyaluronic acid may disrupt cell-cell and cell-scaffold attachments by interrupting intercellular interactions and/or by triggering cellular injury response, which may disrupt intercellular interactions between cells that do not directly contact the hyaluronic acid decoy molecule.

In some embodiments, the decoy molecule may be collagen. Collagen can be isolated in a various forms and from a number of sources. Exemple collagens include collagen type I, collagen type II, collagen type III, collagen type IV, or collagen type V. The collagen can also be fibrillary collagen or non-fibrillar collagen. Low molecular weight collagens can be made, for example, by hydrolysis, and like hyaluronic acid, low molecular weight collagen may disrupt cell-cell and cell-scaffold attachments by interrupting intercellular interactions and/or by triggering cellular injury response, which may disrupt intercellular interactions between cells deeper in the tissue.

In certain embodiments, the decoy molecule may be fibronectin. Fibronectin is a protein dimer, consisting of two nearly identical monomers linked by a pair of disulfide bonds. Fibronectin binds to membrane-spanning receptor proteins called integrins and extracellular matrix components such as collagen, fibrin, and heparin sulfate proteoglycans. Like hyaluronic acid and collagen, fibronectin fragments may disrupt cell-cell and cell-scaffold attachments by interrupting intercellular interactions and/or by triggering cellular injury response, which may disrupt intercellular interactions between cells deeper in the tissue.

In some embodiments, the decoy molecule may be elastin. Elastin is a protein found in connective tissue and allows many tissues in the body to resume their shape after stretching or contracting. Like hyaluronic acid, collagen, and fibronectin, elastin fragments may disrupt cell-cell and cell-scaffold attachments by interrupting intercellular interactions and/or by triggering cellular injury response, which may disrupt intercellular interactions between cells deeper in the tissue.

The compositions of various embodiments may include nearly any active agent, including agents for systemic or local delivery. Non-limiting examples of active agents include a biologic, therapeutic peptides, biomimetic peptide, and small molecule and macromolecular analgesic agents, antifungal agents, antibacterial agents, anesthetic agents, and steroids.

Biologic, therapeutic peptides, and biomimetic peptide encompassed by embodiments include, but are not limited to, botulinum toxin and chimeras or derivatives thereof, antibodies, antibody fragments, derivatives of antibodies, Rejuline, CG-Purilux, CG-Dermaheal, CGKeramin2, Prohairin-B4, CG-TGP2, CG-EDP3, CG-IDP, and the like and combinations thereof. Further examples can be found in US2014/0309157, which is related to peptides for promotion of hair growth and WO 2015/17601, which describes peptides having antioxidant activity or that

Non-limiting examples of analgesic agents, antifungal agents, antibacterial agents, and anesthetic agents, and steroids include gabapentin, pregabalin, minocycline, acetyl salicylic acid, cyclosporine, tacrolimus (FK506), bimatoprost and other PGE2 inhibitors, tadalafil, clindamycin, cortisone, minoxidil, minoxidil sulfate, niacinamide, methyl salicylate, gabapentin, hydrocortisone, palmitoyl-KTTKS peptide, phenytoin, vitamin B12, cyclobenzaprine, anastrozole, lidocaine, retinoic acid, retinyl propionate, minocycline, gentamicin sulfate, bimatoprost, minoxidil sulfate, clobetasol propionate, ascorbic acid, tranexamic acid, salicylic acid (sodium salicylate), hydroquinone, Renokin®, tolfnaftate, clotrimazole, terbinafine, isotretinoin, trentinoin, kojic acid, prednisone, a sunscreen actives such as homosalate, octisalate, octocrylene, or avobenzone, hydrocortisone, lidocaine, ixekizumab taltz, aminolevulinic acid (ALA), baricitinib, tofacitinib, adalimumab, citronella oil, 3(N-butyl-N-acetyl)aminopropionic acid ethyl ester, sarecycline, D3 analogs, calcineurin inhibitors, meclorethamine, immunization antigens, imiquimod, ibuprofen, celecoxib, diclofenac, sildenafil, cyclopyrox, sarecycline, estrogen, conjugated estrogens (PREMARIN®), and the like and combinations thereof.

In various embodiments, the active agent may be one or more of the following: α-Tocopherol, β-Carotene, 2-Mercaptob enzothiazole, Abacavir, Abatacept, Abciximab, Abrotanum, Absinthium, Acacia, Acamprosate, Acarbose, Acebutolol, Acepromazine Maleate, Acetagesic, Acetaminophen, Acetazolamide, Acetic Acid, Acetohydroxamic Acid, Acetylcysteine, Acetyl-Tyrosine, Acidulated Phosphate Fluoride, Acitretin, Aclidinium, Aconite, Aconitum Napellus, Acremonium Cephalosporium, Actaea Spicata, Acyclovir, Adalimumab, Adapalene, Adenine, Adenosine, Adonis Vernalis, Adrenalinum, Aesculus Hip, Aethusa Cynapium, Afatinib, Afoxolaner, Agaricus Muscarius, Agnus Castus, Ailanthus Glandulosus, Aklomide, Alanine, Albendazole, Albiglutide, Albumin Human, Albuterol, Alcaftadine, Alclometasone, Aldesleukin, Alendronate, Aletris Farinosa, Alfalfa, Alfaxalone, Alfentanil, Alfuzosin, Alirocumab, Aliskiren Hemifumarate, Alitretinoin, Allantion, Allopurinol, Almotriptan, Alnus Glutinosa, Aloe, Alosetron, Alprazolam, Alprostadil, Alstonia Constricta, Alternaria Tenuis, Altrenogest, Aluminum, Amantadine, Ambrergris, Ambrosia Artemisiaefolia, Amikacin, Amiloride, Aminocaproic Acid, Aminohippurate, Aminolevulinic Acid, Aminopentamide, Aminophylline, Aminopropazine, Amiodarone, Amitriptyline, Amlodipine, Ammonia, Amobarbital, Amoxapine, Amoxicillin, Amphetamine, Amphomycin, Amphotericin B, Ampicillin, Amprolium, Amyl Nitrosum, Anagallis Arvensis, Anagrelide, Anastrozole, Anhydrous, Anidulafungin, Anthralin, Apomorphine, Apraclonidine, Apramycin, Argatroban, Argentum Metallicum, Arginine, Aripiprazole, Armodafinil, Arnica, Arsenamide, Arsenic, Arsenicum, Artemether, Articaine, Asafoetida, Asarum Europaeum, Asclepias Tuberose, Ascorbic Acid, Asenapine Maleate, Aspartic Acid, Aspirin, Atracurium Besylate, Atriplex Lentiformis, Atropa Belladonna, Atropine, Attapulgite, Aureobasidium Pullularia Pullulans, Aurum Bromatum, Aurum Iodatum, Aurum Metallicum, Aurum Muriaticum, Avena Sativa, Avibactam, Avilamycin, Avobenzoe, Avovenzone, Axitinib, Azacitidine, Azaperone, Azathioprine, Azelaic Acid, Azelastine, Azithromycin, Aztreonam, Bacitracin, Baclofen, Badiaga, Balsalazide, Balsamum Peruvianum, Bambermycins, Baptisia Tinctoria, Barium, Baryta, Basiliximab, Beclomethasone, Belatacept, Benazepril, Bendroflumethiazide, Bentoquatam, Benzalkonium, Benzocaine, Benzonatate, Benzophenone, Benzoyl peroxide, Benzphetamine, Benztropine, Benzyl Alcohol, Beractant, Beta Carotene, Beta-Aminopropionitrile, Betamethasone, Betaxolol, Bethanechol, Bexarotene, Bezlotoxumab, Bicalutamide, Bicisate, Bimatoprost, Biotin, Bisacodyl, Bismuthum Metallicum, Bisoprolol Fumarate, Bivalirudin, Bleomycin, Boceprevir, Boldenone, Borax, Boricum Acidum, Bosutinib, Botrytis Cinerea, Botulinum Toxin Type A, Bovine Somatotropin (Sometribove Zinc), Brimonidine, Brinzolamide, Brodalumab, Bromfenac, Bromine, Bromocriptine, Budesonide, Bultabital, Bumetanide, Bupivacaine, Buprenorphine, Buquinolate, Buspirone, Butabarbital, Butacaine, Butalbital, Butamben, Butamisole, Butenafine, Butorphanol, Butyl, Cabazitaxel, Cabergoline, Caladium Seguinum, Calamine, Calcarea Acetica, Calcarea Arsenicica, Calcarea Carbonica, Calcarea Caustica, Calcarea Flour, Calcarea Fluorica, Calcarea Iodata, Calcarea Muriatica, Calcarea Oxalica, Calcarea Phosphorica, Calcarea Silicate, Calcarea Suflruica, Calcarea Sulphurica, Calceria Carbonica, Calceria Phosphorica, Calcipotriene, Calcipotriene, Calcitriol, Calcium, Calgest, Cambendazole, Camphor, Canakinumab, Candesartan Cilexetil, Cantharidinum, Cantharis, Capecitabine, Capromorelin, Capsaicin, Capsicum, Captopril, Caramiphen Edisylate, Carbachol, Carbadox, Carbamazepine, Carbamide, Carbidopa, Carbo Animalis, Carbo Vegetabilis, Carbolicum Acidum, Carbomycin, Carbon, Carbonate Lime, Carbonate of Barium, Carbonate Of Potassium, Carbonate Of Sodium, Carboneum, Carboplatin, Carboprost Tromethamine, Carboxymethylcellulose, Carduus Marianus, Carfentanil Citrate, Carisoprodol, Carnidazole, Carprofen, Carum Carvi, Carvedilol, Cascarilla, Casein, Caspofungin, Castanea Vesca, Castoreum, Caulophyllum, Causticum, Cedron, Cefaclor, Cefadroxil, Cefazolin, Cefdinir, Cefepime, Cefotaxime, Cefotetan, Cefovecin, Cefoxitin, Cefpodoxime Proxetil, Cefprozil, Ceftaroline Fosamil, Ceftazidime, Ceftiofur, Ceftriaxone, Cefuroxime, Celecoxib, Cenchris Contortrix, Cephalanthus Occidentalis, Cephalexin, Cephapirin B, Ceritinib, Cetirizine, Cetylpyridinium, Cevimeline, Chaetomium Globosum, Chelidonium Majus, Chenodiol, Chenopodium Anthelminticum, Chimaphila Umbellata, China Sulphuricum, Chinchona Officinalis, Chininum, Chlophedianol, Chloral, Chloramine, Chloramphenicol, Chlorcyclizine, Chlordiazepoxide, Chlorhexidine, Chlorine, Chlorinum, Chlorobutanol, Chloroprocaine, Chloroquine, Chlorothiazide, Chloroxylenol, Chlorphenesin, Chlorpheniramine, Chlorpromazine, Chlorpropamide, Chlortetracycline, Chlorthalidone, Chlorzoxazone, Cholecalciferol, Cholesterinum, Cholestyramine, Choriogonadotropin Alfa, Chorionic Gonadotropin, Chromic, Chromium, Chymotrypsin, Ciclopirox, Cicuta Virosa, Cilastatin, Cilostazol, Cimetidine, Cimex Lectularius, Cimicifuga Racemosa, Cina, Cineraria Maritima, Ciprofloxacin, Cisatracurium, Cisplatin, Cistus Canadensis, Citalopram, Citric Acid, Citroma Magnesium, Cladosporium Cladosporioides, Cladribine, Clarithromycin, Clavulanate, Clemastine, Clematis Erecta, Clenbuterol, Clidinium, Clindamycin, Clioquinol, Clobetasol, Clocortolone, Clodronate, Clofarabine, Clomiphene, Clomipramine, Clonazepam, Clonidine, Clopidogrel, Clopidol, Cloprostenol, Clorazepate, Clorsulon, Clotrimazole, Cloxacillin, Clozapine, Cobalamin, Cobaltum, Cobicistat, Cocaine, Codeine, Colchicine, Colchicinum, Colestipol, Colistimethate, Collagenase Santyl, Colloidal Ferric Oxide, Colloidal Sulfur, Colocynthis, Compound Benzoin, Condurango, Conium, Conjugated Estrogens, Convallaria Majalis, Copaiva Officinalis, Copper, Corticotropin, Cortisone, Cosyntropin, Coumaphos, Cratageus Oxycantha, Cresol, Crizotinib, Crocus Sativus, Cromolyn, Crotalus Horridus, Crotamiton, Croton Tiglium, Crypthecodinium Cohnii DHA Oil, Cubeba Officinalis, Cucurbita Citrullus, Culex Pipiens, Cupric, Cuprum, Curvularia Inaequalis, Cuttlefish Ink, Cyanocobalamin, Cyclamen Europaeum, Cyclizine, Cyclobenzaprine, Cyclophosphamide, Cycloserine, Cyclosporine, Cyproheptadine, Cysteine, Cytarabine, Cythioate, Dabigatran, Dabrafenib, Dacarbazine, Daclatasvir, Daclizumab, Daikon, Dalfampridine, Dalteparin, Danazol, Danofloxacin, Dantrolene, Dapagliflozin, Dapsone, Daptomycin, Darifenacin, Dasabuvir, Dasatinib, Datura Stramonium, Daunorubicin, Decitabine, Decoquinate, Deferasirox, Deferoxamine, Delavirdine, Delphinium Staphisagria Seed, Delsym, Demeclocycline, Deracoxib, Desflurane, Desipramine, Desirudin, Desloratadine, Deslorelin, Desmopressin, Desogestrel, Desonide, Desoximetasone, Desoxycorticosterone, Desvenlafaxine, Dexamethasone, Dexmedetomidine, Dexrazoxane, Dextran, Dextrmethorphan, Dextroamphetamine, Dextrose, Diatrizoate, Diazepam, Diazoxide, Dibasic, Dibucaine, Dichlorophene, Dichlorvos, Dichromate, Diclazuril, Diclofenac, Dicloxacillin, Dicyclomine, Didanosine, Diethylcarbamazine, Diethylpropion, Diflorasone, Difloxacin, Diflunisal, Digitalis Purpurea, Digoxin, Dihydroergotamine, Dihydrostreptomycin, Diltiazem, Dimenhydrinate, Dimethicone, Dimethyl, Dinoprostone, Dioscorea Villosa, Dioscoreinum, Diphenhydramine, Dipiperazine, Diprenorphine, Dipyridamole, Dirlotapide, Disopyramide, Disulfiram, Dithiazanine, Divalproex, Dobutamine, Docetaxel, Docone, Doconexant, Docosanol, Dofetilide, Dog Epithelia, Dog Fennel, Dolasetron, Dolichos Pruriens, Domperidone, Donepezil, Dopamine, Doramectin, Dorzolamide, Doxapram, Doxazosin, Doxepin, Doxercalciferol, Doxorubicin, Doxycycline, Drechsl era Helminthosporium, Dronabinol, Dronedarone, Droperidol, Drosera Rotundifolia, Drospirenone, D-Thiamine, Dulaglutide, Dulcamara, Duloxetine, Durezol, Dutasteride, Dyclonine, Ecamsule, Echinacea Purpurea, Echothiophate, Econazole, Efavirenz, Efinaconazole, Eflornithine, Efrotomycin, Elaps Corallinus, Elbasvir, Eletriptan, Elm Chinese, Eltrombopag Olamine, Embutramid, Emedastine, Emodepside, Empagliflozin, Emtricitabine, Enalaprilat, Enfuvirtide, Enoxaparin, Enrofloxacin, Ensulizole, Entacapone, Entecavir, Enzalutamide, Ephedrine, Ephedrine, Ephedrine, Epicoccum Nigrum, Epigaea Repens, Epinastine, Epinephrine, Epiphegus Virginiana, Epirubicin Hydrochloride, Eplerenone, Epoprostenol, Eprinomectin, Eprosartan, Epsiprantel, Eptifibatide, Equine Thymocyte Immune Globulin, Equisetum Arvense, Equisetum Hyemale, Ergocalciferol, Ergotamine Tartrate, Erigeron Canadensis, Ertapenem, Erysimum Cheiri, Erythromycin, Escitalopram, Esium, Esmolol, Esomeprazole, Estazolam, Esterified, Estradiol, Estrogens, Estrone, Estropipate, Eszopiclone, Ethacrynic Acid, Ethambutol, Ethinyl Estradiol, Ethionamide, Ethopabate, Ethosuximide, Ethyl Alcohol, Ethylisobutrazin, Ethynodiol, Etidronate, Etodolac, Etomidate, Etonogestrel, Etoposide, Etorphine, Eugenia Jambosa, Eugenol, Euphorbia Lathyris, Everolimus, Exemestane, Exenatide, Extract Arisaema Triphyllum Root, Extract Aristolochia Clematitis, Extract Arizona Ash, Extract Arizona Cypress, Extract of Baptisia Tinctoria Root, Extract of Corallium Rubrum, Extract of Abies Canadensis, Extract of Abrus Precatorius Seed, Extract of Anacardium Occidentale, Extract of Anacardium Orientale, Extract of Anamirta Cocculus Seed, Extract of Artemisia Cina Pre-Flowering Top, Extract of Artemisia Vulgaris, Extract of Arum Triphyllum, Extract of Ash Arizona, Extract of Ash White, Extract of Asparagus, Extract of Aspen, Extract of Aspergillus Fumigatus, Extract of Aspergillus Niger, Extract of Australian Pine Beefwood, Extract of Avocado, Extract of Azadirachta Indica, Extract of Bahia Grass, Extract of Bald Cypress, Extract of Barberry, Extract of Barley Food, Extract of Bayberry Wax Myrtle, Extract of Bee Venom, Extract of Beech, Extract of Beef, Extract of Belladonna Leaf, Extract of Bellis, Extract of Berberis Aquarius, Extract of Berberis Aquifolium, Extract of Berberis Vulg, Extract of Berberis Vulgaris, Extract of Bermuda Grass, Extract of Betula Alba, Extract of Birch Black, Extract of Birch River Red, Extract of Birch White, Extract of Bitter Cucumber, Extract of Black Cohosh, Extract of Black Lead, Extract of Black Locust, Extract of Black Pepper, Extract of Black Pollen Walnut, Extract of Black Willow, Extract of Blatta Americana, Extract of Blatta Orientalis, Extract of Blattella Germanica, Extract of Bluegrass Annual, Extract of Box Elder Ash Leaf Maple, Extract of Brazil Nut, Extract of Broccoli, Extract of Brome Grass, Extract of Bryonia, Extract of Buckwheat, Extract of Bushmaster Snake Venom, Extract of Buttercup, Extract of Cabbage, Extract of Cactus Grandiflorus, Extract of Cadmium Sulphuricum, Extract of Caffeine, Extract of Calcitonin Salmon, Extract of Calendula, Extract of California Live Oak Coast, Extract of California Pepper Tree, Extract of California Walnut Black Pollen, Extract of Calomel, Extract of Calotropis Gigantea, Extract of Candida Albicans, Extract of Candida Parapsilosis, Extract of Cantaloupe, Extract of Carelessweed, Extract of Carrot, Extract of Castor Birch, Extract of Castor Equi, Extract of Castor Oil, Extract of Cat Hair, Extract of Cat Pelt, Extract of Cattle Epithelium, Extract of Ceanothus Americanus, Extract of Cedar Elm, Extract of Cedar Mountain, Extract of Cedar Red, Extract of Celery, Extract of Chamomile Plant, Extract of Chastetree, Extract of Cherry, Extract of Chicken Meat, Extract of Chinese Elm, Extract of Chionanthus Virginica, Extract of Cinchona, Extract of Cinnamon, Extract of Citrullus Colocynthis Fruit, Extract of Clam, Extract of Club Moss, Extract of Coal Tar, Extract of Cocculus Cacti, Extract of Cocculus Indicus, Extract of Cocklebur, Extract of Cocoa Bean Whole Bean Chocolate, Extract of Cocoa Butter, Extract of Coconut, Extract of Codfish, Extract of Coffea, Extract of Collinsonia Canadensis, Extract of Collinsonia Canadensis Root, Extract of Colloidal Oatmeal, Extract of Comfrey Plant, Extract of Comfrey Root, Extract of Common Mugwort, Extract of Common Sagebrush, Extract of Common Wormwood Annual, Extract of Conium Maculatum, Extract of Coral Snake (Micrurus Fulvius) Immune Globulin Antivenin (Equine), Extract of Corn, Extract of Cotton Linters, Extract of Cottonseed, Extract of Cottonwood Eastern Common, Extract of Cottonwood Fremont, Extract of Cottonwood Western, Extract of Crab, Extract of Cramp Bark, Extract of Cucumber, Extract of Cuttlefish, Extract of Cypress Arizona, Extract of Cypress Bald, Extract of Daisy, Extract of Dandelion, Extract of Daphne Indica, Extract of Daphne Mezereum Bark, Extract of Deadly Nightshade, Extract of Dock Sour Sheep Sorrel, Extract of Dock Yellow, Extract of Eastern Cottonwood Common, Extract of Echinacea Angustafolia, Extract of Echinacea Angustifolia, Extract of Egg White, Extract of Egg Yolk, Extract of Elm American, Extract of Elm Cedar, Extract of Elotuzumab, Extract of English Plantain, Extract of English Walnut, Extract of English Walnut Pollen, Extract of Eucalyptus, Extract of Eucalyptus Oil, Extract of Eupatorium Perfoliatum, Extract of Eupatorium Perfoliatum Flowering Top, Extract of Eupatorium Purpureum, Extract of Euphrasia, Extract of European Elder, Extract of False Ragweed Bur, Extract of Flounder, Extract of Fragrant Sumac, Extract of Fraxinus Americana, Extract of Fremont Cottonwood, Extract of Freshwater Sponge, Daisy, Extract of Fucus Vesiculosus, Extract of Galphimia Glauca Flowering Top, Extract of Garden Rue, Extract of Garlic, Extract of Gelsemium Sempervirens, Extract of Gelsemium Sempervirens Root, Extract of Geranium Maculatum, Extract of German Cockroach, Extract of Ginger, Extract of Ginkgo Biloba, Extract of Goat Milk, Extract of Goldenrod, Extract of Goldenseal, Extract of Gopher plant, Extract of Grapefruit, Extract of Graphite, Extract of Green Coffee, Extract of Green Pea English, Extract of Guinea Pig Epithelia, Extract of Hackberry, Extract of Hazelnut Pollen, Extract of Heloderma Horridum Venom, Extract of Hemoglobin Glutamer-200 (bovine), Extract of Honey Bee, Extract of Honeydew, Extract of Hops, Extract of Horse Chestnut, Extract of Horse Epithelia, Extract of Horsetail, Extract of Indian Cockle, Extract of Ipecac, Extract of Ipecac Root, Extract of irginia Live Oak, Extract of Iris Germanica Root, Extract of Italian Rye Grass, Extract of Jalapa, Extract of Johnson Grass, Extract of Juglans Regia, Extract of Juniper Western, Extract of Juniperus Communis, Extract of Juniperus Sabina Leafy Twig, Extract of Juniperus Virginiana, Extract of Lachesis Muta, Extract of Lamb, Extract of Lima Bean, Extract of Lobster, Extract of Locust Black Non Stock, Extract of Loose Wheat Smut, Extract of Magnolia Grandiflora, Extract of Maple Red, Extract of Maple Sugar, Extract of Marking Nut, Extract of Marshelder Burweed, Extract of Marshelder Rough, Extract of Matricaria Recutita, Extract of Meadow Fescue Grass Standardized, Extract of Melaleuca Pollen, Extract of Melilotus Officinalis, Extract of Melissa Officinalis, Extract of Mexican Tea, Extract of Milk of Magnesia, Extract of Milk Thistle, Extract of Milk Whole Cows, Extract of Mountain Arnica, Extract of Mountain Cedar, Extract of Mountain Tobacco, Extract of Mouse Epithelia, Extract of Mouse Epithelium, Extract of Mucor Circinelloides F. Lusitanicus, Extract of Mucor Plumbeus, Extract of Mucor Racemosus, Extract of Mugwort Common, Extract of Mulberry Red, Extract of Mulberry White, Extract of Mustard Seed, Extract of Oak California Live Coast, Extract of Oak Red, Extract of Oak Virginia Live, Extract of Oak White, Extract of Oat Grain, Extract of Oat Straw, Extract of Oat Wild Pollen, Extract of Oatmeal, Extract of Oatstraw, Extract of Oil of Mustard Seed, Extract of Old Balsam, Extract of Oleander, Extract of Olive, Extract of Olive Pollen, Extract of Onion, Extract of Orange, Extract of Orchard Grass, Extract of Orris Root, Extract of Oyster, Extract of Palm Queen Coco Palm, Extract of panish Fly, Extract of Parsley, Extract of Passiflora Incarnata, Extract of Passiflora Incarnata Top, Extract of Passion Flower, Extract of Peach, Extract of Peanut, Extract of Pear, Extract of Pecan, Extract of Pecan Pollen, Extract of Pectin, Extract of Pepper Tree California, Extract of Periplaneta Americana, Extract of Picea Mariana Resin, Extract of Pigweed Rough Redroot, Extract of Pigweed Spiny, Extract of Pine Australian Beefwood, Extract of Pine White, Extract of Pine Yellow, Extract of Pineapple, Extract of Pinto Bean Kidney Bean, Extract of Pinus Lambertiana, Extract of Pinus Sylvestris, Extract of Pistachio Nut, Extract of Plantago Major, Extract of Plantago Seed, Extract of Plantain English, Extract of Plum, Extract of Poison Hemlock, Extract of Poison Ivy, Extract of Poison Nut, Extract of Poison oak, Extract of Pongia Officinalis Skeleton, Extract of Poplar White, Extract of Pork, Extract of Pot Marigold, Extract of Prairie Sage, Extract of Psyllium, Extract of Pure Flint, Extract of Purple Cone Flower, Extract of Quack Grass, Extract of Quebracho, Extract of Queen Palm Coco Palm, Extract of Quercus Glandium Spiritus, Extract of Rabbit, Extract of Rabbit Epithelium, Extract of Ragweed False Bur, Extract of Ragweed Short, Extract of Ragweed Slender, Extract of Ragweed Southern, Extract of Ragweed Tall Giant, Extract of Ragweed Western, Extract of Rancid Beef, Extract of Ranunculus Bulbosus, Extract of Raw Opium Gum, Extract of Red Cedar, Extract of Red Maple, Extract of Red Mulberry, Extract of Red Oak, Extract of Red Onion, Extract of Redtop Grass, Extract of Rhamnus Frangula, Extract of Rhododendron Aureum Leaf, of Rhododendron Chrysanthum, Extract of Rhodotorula Rubra, Extract of Rhubarb, Extract of River Birch Red, Extract of Robinia Pseudoacacia, Extract of Rough Marshelder, Extract of Rough Pigweed, Extract of Rough Pigweed Redroot, Extract of Rumex, Extract of Russian Thistle, Extract of Ruta, Extract of Rye, Extract of Rye Grass, Extract of Rye Grass Italian, Extract of Sage Prairie, Extract of Sagebrush Common, Extract of Salmon, Extract of Salt Grass, Extract of Salvia Officinalis, Extract of Sambucus, Extract of Sanguinaria Canadensis, Extract of Saponaria Officinalis Root, Extract of Schoenocaulon Officinale Seed, Extract of Senecio, Extract of Senna, Extract of Sepia, Extract of Serum Gonadotropin, Extract of Sesame Seed, Extract of Shagbark Hickory, Extract of Short Ragweed Pollen Allergen Extract, Extract of Shrimp, Extract of Slender Ragweed, Extract of Solanum, Extract of Solidago Virgaurea, Extract of Solidago Virgaurea Flowering Top, Extract of Sour Dock Sheep Sorrel, Extract of Southern Ragweed, of Soybean, Extract of Soybean Oil, Extract of Spinach, Extract of Spiny Pigweed, Extract of Spongia Officinalis Skeleton, Extract of Squash, Extract of St Ignatius Bean, Extract of St Johns Wort, Extract of Stemphylium Solani, Extract of Stinging Nettle, Extract of Strawberry, Extract of String Bean Green Bean, Extract of Strychnos Ignatii Seed, Extract of Strychnos Nux-Vomica Seed, Extract of Sugar Maple, Extract of Sweet Corn, Extract of Sweet Potato, Extract of Sweet Vernal Grass Standardized, Extract of Sweetgum, Extract of Sweetgum Non Stock, Extract of Sycamore American, Extract of Symphytum, Extract of Tarentula Cubensis, Extract of Tarentula Hispana, Extract of Thuja OCC, Extract of Tobacco Leaf, Extract of Tomato, Extract of Tuna, Extract of Turkey Meat, Extract of Turpentine, Extract of Turpentine Oil, Extract of Uva Ursi, Extract of Valerian, Extract of Vanilla, Extract of Vegetable Charcoal, Extract of Velvet Grass, Extract of Veratrum Album, Extract of Veratrum Album Root, Extract of Veratrum Viride, Extract of Verbascum Thapsus, Extract of Verbena Hastata, Extract of Viburnum Opulus, Extract of Viburnum Opulus Root, Extract of Viola Odorata, Extract of Viola Tricolor, Extract of Walnut Black Pollen, Extract of Walnut California Black Pollen, Extract of Walnut English Pollen, Extract of Water Hemp, Extract of Watermelon, Extract of Western Cottonwood, Extract of Western Juniper, Extract of Western Ragweed, Extract of Wheat Pollen, Extract of Wheat Smut, Extract of White Alder, Extract of White Ash, Extract of White Birch, Extract of White Cedar, Extract of White Mulberry, Extract of White Oak, Extract of White Oxide Of Arsenic, Extract of White Petrolatum, Extract of White Petrolatum Mineral Oil, Extract of White Pine, Extract of White Poplar, Extract of White Potato, Extract of White Seedless Grape, Extract of Whole Arnica Plant, Extract of Whole Egg, Extract of Whole Wheat Wheat Grain, Extract of Wild Hops, Extract of Wild Lavender, Extract of Wild Pollen Oat, Extract of Willow Black, Extract of Wind Flower, Extract of Witch Hazel, Extract of Wood Creosote, Extract of Woody Nightshade, Extract of Wormseed, Extract of Wormwood Common Annual, Extract of Wyethia Helenioides, Extract of Wyethia Helenioides Root, Extract of Yeast Saccharomyces Cerevisiae, Extract of Yellow Dock, Extract of Yellow Jasmine, Extract of Yellow Pine, Extrat of Protortonia Cacti, Ezogabine, Fagopyrum Esculentum, Famciclovir, Famotidine, Famphur, Febantel, Fel Tauri, Felbamate, Felodipine, Fenbendazole, Fenofibrate, Fenofibric Acid, Fenoldopam, Fenoprofen, Fenprostalene, Fentanyl, Ferric, Ferrous Fumarate Fire Ant, Ferrous Fumarate Fish Berry, Fesoterodine, Fexofenadine, Fibrinogen, Ficus Religiosa, Filix Mas, Finasteride, Fingolimod, Firocoxib, Flavone, Flecainide, Florbetapir, Florfenicol, Fluconazole, Flucytosine, Fludarabine, Fludeoxyglucose, Fludrocortisone, Flumazenil, Flumethasone, Flunisolide, Flunixin, Fluocinonide, Fluorescein, Fluoride, Fluorometholone, Fluorouracil, Fluoxetine, Fluoxymesterone, Fluphenazine, Fluprostenol, Fluralaner, Flurandrenolide, Flurazepam, Flurbiprofen, Flutamide, Fluticasone, Fluvastatin, Fluvoxamine, Foeniculum Vulgare, Folic Acid, Follitropin, Fomepizole, Formaldehyde, Formalin, Formic Acid, Formica Rufa, Formoterol, Fosaprepitant Dimeglumine, Foscarnet, Fosfomycin Tromethamine, Fosinopril, Fosphenytoin, Frovatriptan, Fulvestrant, Furazolidone, Furosemide, Fusarium, Gabapentin, Gadobenate, Gadodiamide, Gadoteridol, Gadoversetamide, Galantamine, Galanthus Nivalis, Gallicum Acidum, Gallium, Gambogia, Gamithromycin, Ganciclovir, Ganirelix, Gatifloxacin, Gauifenesin, Gaultheria Procumbens, Gefitinib, Gelatin, Gemcitabine, Gemfibrozil, Gentamicin, Gentiana Quinqueflora, Glatiramer, Gleptoferron, Glimepiride, Glipizide, Glonoinum, Glucagon, Gluconolactone, Glutamic Acid, Glutathione, Glyburide, Glycerin, Glycine, Glycopyrrolate, Glycyrrhiza Glabra, Gnaphalium, Goldenseal Root, Gonadorelin, Gonadorelin Acetate, Gonadotropin Releasing Factor—Diphtheria Toxoid Conjugate, Goserelin, Gossypium Herbaceum, Gramicidin, Granisetron, Grapiprant, Gratiola Officinalis, Grazoprevir, Griseofulvin, Guaco, Guafenesin, Guaiacol, Guaiacum, Guaifenesin, Guaifensin, Guanfacine, Haemophilus b Conjugate Vaccine (Meningococcal Protein Conjugate), Hahnemanns Causticum, Halcinonide, Halobetasol, Halofuginone, Haloperidol, Halothane, Haloxon, Hamamelis, Hedeoma Pulegioides, Hekla Lava, Helianthus Annuus, Heliox, Helium, Helleborus Foetidus, Helleborus Niger, Helminthmucor, Helonias Dioica, Heme Iron Polypeptide, Henbane, Hepar, Heparin, Heptahydrate, Hetacillin, Hetastarch, Hexachlorophene, Hexaminolevulinate, Histamine, Histidine, Homatropine, Homosalate, Human Insulin, Human Papillomavirus 9-Valent Vaccine, Human Papillomavirus Quadrivalent (Types 6, 11, 16, 18) Vaccine, Human Recombinant, Human Rho(D) Immune Globulin, Humulus Lupulus, Hyaluronate, Hyaluronidase, Hydorcortisone, Hydralazine, Hydrangea Arborescens, Hydrastis Canadensis, Hydrochloride, Hydrochlorothiazide, Hydrocodone, Hydrocortisone, Hydrocotyle Asiatica, Hydrofluoric Acid, Hydrogen, Hydrogenate Palm Kernel Oil, Hydromorphone, Hydroquinone, Hydrous, Hydroxocobalamin, Hydroxychloroquine, Hydroxyethyl, Hydroxyurea, Hydroxyzine, Hygromycin B, Hyoscyamine, Hyoscyamus Niger, Hypericum, Hypromellose, Ibandronate, Iberis amara, Ibuprofen, Ibutilide, Ichthyolum, Icodextrin, Icosapent, Idarubicin, Idarucizumab, Ifosfamide, Ignatia Amara, Ignatius Bean, tins Versicolor, Iloperidone, Imatinib, Imidacloprid, Imidocarb, Imipenem, Imipramine, Imiquimod, Immune Globulin (Human), Impure Calcium, Incobotulinumtoxina, Indacaterol, Indapamide, Indigo, Indinavir, Indium, Indomethacin, Infliximab, Influenza Virus Vaccine, Influenzinum, Ingenol, Insulin, Interferon, Iodides Tincture, Iodinated Casein, Iodine, Iodipamide Meglumine, Iodium, Iodixanol, Iodochlorhydroxyquin, Iohexol, Iopamidol, Iothalamate, Ioversol, Ipecacuanha, Ipilimumab, Ipratropium, Irbesartan, Iridium, Irinotecan, Tenax, Iris Versicolor, Iron, Isavuconazonium, Isodium, Isoflupredone, Isoflurane, Isoleucine, Isometheptene, Isoniazid, Isopropamide, Isopropyl Alcohol, Isoproterenol, Isosorbide, Isotretinoin, Isradipine, Itraconazole, Ivermectin, Ixabepilone, Ixekizumab, Jacaranda Caroba, Jacobaea Maritima, Justicia Adhatoda, Kali Arsenicosum, Kali Arsenicum, Kali Bechromate, Kali Bechromate Karaya Gum Bassora, Kali Bechromate Kentucky Bluegrass (June) Standardized, Kali Bechromate Kochia Firebush, Kali Bechromate Krameria Lappacea Root, Bechromate Lemon, Kali Bechromate Leopards Bane, Kali Bechromate Lettuce, Kali Bichromicum, Kali Bromatum, Kali Carbonate, Kali Carbonicum, Kali Iodatum, Kali Muriaticum, Kali Muriaticum, Silicea, Kali Nitricum, Kali Phosphoricum, Kali Phosphoricum, Kali Sulphuricum, Kali Phosphoricum, Magnesia Phosphorica, Natrum Phosphoricum, Kali Sulph, Kali Sulphuricum, Kalmia Latifolia, Kanamycin Sulfate, Kapok, Ketamine, Ketamine, Ketoconazole, Ketoprofen, Ketorolac, Ketotifen, Ketotifen, Kreosotum, Labetalol, Lac Caninum, Lac Defloratum, Lac Felinum, Lac Vaccinum, Lachnanthes Tinctoria, Lacosamide, Lactic Acid, Lacticum Acidum, Lactuca Virosa, Lactulose, Laidlomycin, Lamium Album, Lamivudine, Lamotrigine, Lanolin, Lanreotide, Lansoprazole, Lapatinib, Lapis Albus, Lappa Major, Lasalocid, Latanoprost, Lathyrus Sativus, Latrodectus Mactans, Lauric Acid, Laurocerasus, Laxative, L-Cysteine, Lead, Lecithin, Ledum, Ledum Palustre, Ledum Palustre Twig, Leflunomide, Lemna Minor, Leptandra Virginica, Lesinurad, Letrozole, Leucine, Leucovorin, Leuprolide, Levalbuterol, Levamisole, Levetiracetam, Levobunolol, Levocarnitine, Levodopa, Levofloxacin, Levoleucovorin, Levomefolate, Levomilnacipran, Levonordefrin, Levonorgestrel, Levorphanol, Levothyroxine, Levulose, Lidocaine, Lilium Tigrinum, Linaclotide, Linagliptin, Lincomycin, Lindane, Linezolid, Linolenic Acid, Liothyronine, Liraglutide, Lisinopril, Lithium, Lixisenatide, Lobaria Pulmonaria, Lobelia Inflata, Lodoxamide Tromethamine, Loperamide, Lopinavir, Loratadine, Lorazepam, Losartan, Loteprednol, Lovastatin, Loxapine, Lubiprostone, Lufenuron, Luffa Operculata, Lugols, Luliconazole, Lumefantrine, Luprostiol, Lutein, Lycopodium, Lycopus Virginicus, Lysine, Lytta Vesicatoria, Macrocrystalline, Maduramicin Ammonium, Mag Phos, Magnesium, Malathion, Manganese, Manganum, Mannitol, Maprotiline Hydrochloride, Maraviroc, Marbofloxacin, Maropitant, Maxzide, Mebendazole, Mebrofenin, Mecamylamine, Mecasermin, Mechlorethamine, Meclizine, Meclofenamate, Medetomidine, Medroxyprogesterone, Mefenamic Acid, Mefloquine, Megestrol Acetate, Melarsomine, Melatonin, Melengestrol Acetate, Meloxicam, Melphalan, Memantine, Mentha Piperita, Menthol, Menyanthes Trifoliata, Mepenzolate, Meperidine, Mephitis Mephitica, Mepivacaine H, Mepolizumab, Meprobamate, Meradimate, Mercaptopurine, Mercurius Corrosivus, Mercurius Dulcis, Mercurius Iodatus Flavus, Mercurius Iodatus Ruber, Mercurius Solubilis, Mercurous, Mercury, Meropenem, Mertiatide, Mesalamine, Mesna, Mesquite, Metaxalone, Metformin, Methadone, Methamphetamine, Methazolamide, Methenamine, Methimazole, Methionine, Methocarbamol, Methotrexate, Methoxsalen, Methoxy Polyethylene Glycol-Epoetin Beta, Methscopolamine, Methsuximide, Methyclothiazide, Methyl Salicyl ate, Methyldopa, Methylene Blue, Methylergonovine Maleate, Methylphenidate, Methylprednisole, Methylprednisolone, Methylsalicylate, Methyltestosterone, Metoclopramide, Metolazone, Metoprolol, Metoserpate, Metronidazole, Mexiletine, Mezereum, Mibolerone, Micafungin, Miconazole, Midazolam, Miglitol, Miglustat, Milbemycin Oxime, Millefolium, Milnacipran, Milrinone, Minocycline, Minoxidil, Mirabegron, Mirtazapine, Misoprostol, Mitomycin, Mitotane, Mitoxantrone, Mivacurium, Modafinil, Moexipril, Molybdenum, Mometasone Furoate, Monensin, Monobasic, Monohydrate, Montelukast, Morantel, Morphine, Morrhuate, Moschus, Moxidectin, Moxifloxacin, Mupirocin, Murex Purpurea, Muriaticum Acidum, Mycophenilic, Mygale, Myrica Cerifera, Myristica Sebifer, Myristyl, Nabilone, Nabumentone, Nadolol, Nafarelin, Nafcillin, Naftifine, Naja Tripudians, Nalbuphine, Nalorphine, Naloxegol, Naloxone, Naltrexone, Nandrolone, Naphazoline, Naphthalinum, Naproxen, Narasin, Naratriptan, Phos Nutmeg, Natamycin, Nateglinide, Natrum, Nebivolol, Necitumumab, Nedocromil, Nefazodone, Nelarabine, Neomycin, Neostigmine, Nepafenac, Nequinate, Neurospora Intermedia, Neutral Sodium Fluoride, Nevirapine, Niacin, Nicarbazin, Nicardipine, Niccolum, Nicotine, Nifedipine, Nigrospora, Nilotinib, Nilutamide, Nimodipine, Nintedanib, Nisoldipine, Nitenpyram, Nitric Acid, Nitrofurantoin, Nitrofurazone, Nitrogen, Nitroglycerin, Nitromide, Nitrous Oxide, Nivolumab, Nizatidine, Norelgestromin, Norepinephrine, Norethindrone, Norgestimate, Norgestomet, Norgestrel, Nortriptyline, Novobiocin, Nux Moschata, Nux vomica, Nystatin, Ocimum Sanctum, Ocitnoxate, Ocitsalate, Oclacitinib, Octinoxate, Octisalate, Octobenzone, Octocrylene, Octreotide, Oenanthe Crocata, Ofatumumab, Ofloxacin, Olanzapine, Olaparib, Olaratumab, Oleate Sodium, Olmesartan Medoxmil, Olodaterol, Olopatadine, Olsalazine, Ombitasvir, Omeprazole, Onabotulinumtoxina, Ondansetron, Onosmodium Virginianum, Oophorinum, Opium, Opuntia Vulgaris, Orbifloxacin, Orgotein, Orlistat, Ormetoprim, Orphenadrine Citrate, Oseltamivir Phosphate, Osimertinib, Osmium Metallicum, Ova Tosta, Ovine Digoxin Immune Fab, Oxacillin, Oxalicum Acidum, Oxaliplatin, Oxandrolone, Oxaprozin, Oxazepam, Oxcarbazepine, Oxctinoxate, Oxibendazole, Oxiconazole, Oxide of Aluminum, Oxybenzone, Oxybutynin, Oxycodone, Oxygen, Oxymetazoline, Oxymorphone, Oxyquinoline, Oxytetracycline, Oxytocin, Paclitaxel, Padimate O, Paeonia Officinalis, Palbociclib, Paliperidone, Palladium Metallicum, Pamabrom, Pamidronate, Pancrelipase, Pancuronium, Panobinostat, Pantoprazole, Pantothenic Acid, Papaverine, Paraffinum, Pareira Brava, Paricalcitol, Paris Quadrifolia, Paritaprevir, Paroxetine, Pasireotide, Pazopanib, Peg-3350, Pegaspargase, Pegbovigrastim, Peginterferon Alfa-2a, Peginterferon Alfa2b, Pegvisomant, Pembrolizumab, Pemetrexed, Penciclovir, Penicillamine, Penicillin G, Penicillin V, Penicillium Chrysogenum, Penicillium Glabrum, Penicillium Roqueforti, Pentavalent, Pentazocine, Pentobarbital, Pentostatin, Pentoxifylline, Perflutren, Pergolide Mesylate, Perindopril Erbumine, Permethrin, Perphenazine, Petrolatum, Petroleum, Petroselinum, Phellandrium Aquaticum, Phenazopyridine, Phendimetrazine, Phenelzine, Pheniramine Maleate, Phenobarbital, Phenol, Phenothiazine, Phenoxybenzamine, Phenozapyridine, Phentermine, Phentolamine, Phenykephrine, Phenyl Salicylate, Phenylalanine, Phenylbenzimidazole Sulfonic Acid, Phenylbutazone, Phenylephrine, Phenylpropanolamine, Phenyltoloxamine, Phenytoin, Phoma Herbarum, Phophorus, Phosmet, Phosphate of Iron, Phosphorated Carbohydrate, Phosphoric Acid, Phosphorus, Physalis Alkekengi, Physostigma Venenosum, Phytolacca Americana Root, Phytolacca Decandra, Phytonadione, Picric Acid, Picricum Acidum, Pilocarpine Hydrochloride, Pilocarpus, Pimecrolimus, Pimobendan, Pindolol, Pioglitazone, Piperacillin, Piperazine, Piperonyl, Pirlimycin, Piroxicam, Pituitary Luteinizing Hormone, Pix Liquida, Platinum, Plerixafor, Plumbum, Podofilox, Podophyllum, Podophyllum Resin, Poloxalene, Polyehtylene, Polymyxin, Polyoxyethylene, Polyporus Pinicola, Polysorbate 80, Polysulfated Glycosaminoglycan, Polyvinyl Alcohol, Ponazuril, Poractant Alfa, Porcine, Posaconazole, Potassium, Pothos Foetidus, Povidone, Pradofloxacin, Pralidoxime Chloride, Pramipexole, Pramlintide, Pramoxine, Prasugrel, Pravastain, Praziquantel, Prazosin, Prednicarbate, Prednisolone, Prednisone, Pregabalin, Prilocaine, Primaquine, Primidone, Privet, Probenecid, Procainamide, Prochlorperazine, Progesterone, Proguanil, Proline, Promazine, Promethazine, Propafenone, Propiopromazine, Propofol, Propoxyphene, Propranolol, Propylene, Propylhexedrine, Propylthiouracil, Prostalene, Protriptyline, Providone Iodine, Prunus Spinosa, Pseudoephedrine, Pullularia Pullulans, Pulsatilla, Pyrantel, Pyrazinamide, Pyrethrum, Pyridostigmine, Pyridoxine, Pyrilamine, Pyrimethamine, Pyrithione Zinc, Pyrogenium, Quassia Amara, Quetiapine, Quinapril, Quinidine, Rabacfosadine, Rabeprazole, Racepinephrine, Ractopamine, Radium, Raloxifene, Raltegravir, Ramipril, Ramucirumab, Ranitidine, Raphanus Sativus, Rasagiline, Rasburicase, Ratanhia, RauwolfiaSerpentina, Recombinant, Regadenoson, Repaglinide, Reserpine, Resorcinol, Retapamulin, Rheum Officinale, Rhodium, RhusGlabra, Rice, Ribavirin, Ribociclib, Riboflavin, RicinusCommunis, Rifabutin, Rifampin, Rifapentine, Riluzole, Rimabotulinumtoxinb, Rimantadine, Rimexolone, Risedronate, Risperidone, Ritonavir, Rivastigmine, Rizatriptan, Robenacoxib, Robenidine, Robinul, Rocuronium, Roflumilast, Romifidine, Ropinirole, Ropivacaine, Rosiglitazone Maleate, Rosuvastatin Calcium, Roxarsone, Rubella, Rubidium, Rue, Sabadilla, Sabal Serrulata, Sabina, Saccharomyces Cerevisiae, Saccharum Lactis, Sacubitril, Salicyclic Acid, Salicylamide, Saline, Salinomycin, Salix Nigra, Salmeterol, Salsalate, Samarium SM 153 Lexidronam, Santoninum, Saquinavir Mesylate, Sarcolacticum AcidumSargramostim, Sarocladium Strictum, Sarolaner, Sarracenia Purpurea, Sarsaparilla, Saxagliptin, Schizochytrium Dha Oil, Scopalamine, Scopolamine, Scrophularia Nodosa, Scutellaria Lateriflora, Secale Cornutum, Secobarbital, Secukinumab, Selamectin, Selan, Selegiline, Selenium, Selenomethionine, Semduramicin, Sennosides, Serine, Sertaconazole, Sertraline, Sevelamer Carbonate, Sevoflurane, Shark Liver Oil, Sildenafil, Silica, Silicon, Silver, Simethicone, Simvastatin, Sinapis Nigra, Sincalide, Sinecatechins, Sirolimus, Sitagliptin, Skatolum, Sodium, Solenopsis Invicta, Somatropin, Sonidegib, Sorbitol, Sotalol, Spectinomycin, Spigelia, Spinosad, Spironolactone, Spongia Tosta, Stannous, Stanozolol, Staphysagria, Starch, Stavudine, Stellaria Media, Sticta Pulmonaria, Stigmata Maidis, Stramonium, Streptomycin, Streptozocin, Strontium, Strophanthus Hispidus, Succinylcholine, Sucralfate, Sufentanil, Sugammadex, Sulbactam, Sulconazole, Sulfabromomethazine, Sulfacetamide, Sulfachlorpyridazine, Sulfadiazine, Sulfadimethoxine, Sulfaethoxypyridazine, Sulfamerazine, Sulfamethazine, Sulfamethizole, Sulfamethoxazole, Sulfanilamide, Sulfanitran, Sulfaquinoxaline, Sulfasalazine, Sulfisoxazole, Sulfomyxin, Sulfur, Sulindac, Sulphide OfAntimony, Sulphur, Sumatriptan, Sumatriptan, Succinate, Sumbul, Sunitinib Malate, Suvorexant, Syzygium Jambolanum, Tabaccum, Tabaccum Tall Ragweed Giant, Tacrolimus, Tadalafil, Talc, Taliglucerase Alfa, Tamoxifen Citrate, Tamsulosin Hydrochloride, Tanacetum Vulgare, Tannic Acid, Tapentadol, Taraxacum Officinalis, Tartaremetic, Tartaricum Acidum, Taurine, Tavaborole, Tazarotene, Tazobactam, Tazobactam, Technetium, Telbivudine, Telithromycin, Tellurium Metallicum, Telmisartan, Temazepam, Temozolomide, Temsirolimus, Tenofovir Disoproxil Fumarate, Tepoxalin, Terazosin, Terbinafine, Terbutaline, Terconazole, Terebinthina, Teriparatide, Testosterone, Tetanus, Tetracaine, Tetracycline, Tetrafluoroborate, Tetrahydrozoline, Tetrakis, Teucrium Marum, Thallium, Thallous, Thaspium Aureum, Thea Sinensis, Thenium Closylate, Theophylline, Theridion, Thiabendazole, Thialbarbitone, Thiamin, Thiamine, Thiamylal, Thiopental, Thioridazine, Thiosinaminum, Thiostrepton, Thiotepa, Thiothixene, Thlaspi Bursa-Pastoris, Threonine, Thrombin Human, Thymol, Thymus Serpyllum, Thyroidinum, Tiagabine, Tiamulin, Ticagrelor, Ticarcillin, Ticlopidine, Tigecycline, Tildipirosin, Tiletamine, Tilia Europaea, Tilmicosin, Tiludronate, Timolol Maleate, Tincture Of Benzoin, Tinidazole, Tioconazole, Tiopronin, Tioxidazole, Tipranavir, Titanium, Tizanidine, Tl 201, Tobramycin, Toceranib, Tocopheryl Acid, Succinate, Tofacitinib, Tolazamide, Tolazoline, Tolbutamide, Tolcapone, Tolmetin, Tolnaftate, Tolterodine, Toluene, Topiramate, Topotecan, Toremifene, Torsemide, ToxicodendronPubescensLeaf, Tramadol, Trametinib, Trandolapril, TranexamicAcid, Tranylcypromine, Travoprost, Trazodone, Trenbolone, Tretinoin, Triamcinolone, Triamterene, Triazolam, Tribasic, TricaieTrichlorfon, Trichlormethiazie, TrichloroaceticAcid, Trichophyton, Triclocarban, Triclosan, Trientine, Trifluoperazine, Trifolium, Pratense, Trifolium, Repens, Trihexyphenidyl, Trilostane, Trimeprazine, Trimethadione, Trimethoprim, Tripelennamine, Tripolidine, Trolamine, Tromethamine, Tropicamid, Trospium, Trypsin, Tryptophan, Tulathromycin, Tylosin, Tylvalosin, Tyrosine, Umeclidinium, Undecylenic Acid, Uranium Nitricum, Urea, Ursodiol, Urtica Urens, Ustilago Maidis, Valacyclovir, Valganciclovir H, Valine, Valproate, Valproic Acid, Valsartan, Vancomycin, Vandetanib, Vardenafil, Varenicline, Vasopressin, Vecuronium B, Venetoclax, Venlafaxine, Vilanterol, Vilazodone, Vinca Minor, Vincristine, Vinorelbine, Virginiamycin, Viscum Album, Vitamin A, Vitamin B6, Vitamin C, Vitamin D, Vitamin D3, Vitamin E, Vorapaxar, Voriconazole, Vorinostat, Wal-Zan, Wal-Zyr, Warfarin, Xanthoxylum Fraxineum, Xray, Xylazine, Yohimbine, Yohimbinum, Zafirlukast, Zaleplon, Zanamivir, Zavara, Zeranol, Zidovudine, Zileuton, Zilpaterol, Zinc, Zingiber Officinale, Ziprasidone, Ziv-Aflibercept, Zoalene, Zolazepam, Zoledronic Acid, Zolmitriptan, Zolpidem, Zonisamide,

In some embodiments, the active agent may be a for veterinary use. Such agents include, but are not limited to, 2-mercaptobenzothiazole, acepromazine maleate, acetazolamide sodium, acetylsalicylic acid, afoxolaner, aklomide, albendazole, albuterol sulfate, alfaxalone, altrenogest, amikacin sulfate, aminopentamide hydrogen sulfate, aminopropazine fumarate, amitraz, ammonium chloride, amoxicillin trihydrate, amphomycin calcium, ampicillin anhydrous, ampicillin sodium, ampicillin trihydrate, amprolium, apramycin sulfate, arsenamide sodium, atipamezole hydrochloride, atropine, attapulgite, avilamycin, azaperone, bacitracin methylene disalicylate, bacitracin zinc, balsam peru oil, bambermycins, beta-aminopropionitrile, betamethason valerate, betamethasone acetate, betamethasone dipropionate, betamethasone sodium phosphate, betamethasone valerate, bismuth subcarbonate, boldenone undecylenate, bovine somatotropin (sometribove zinc), bunamidine hydrochloride, bupivacaine, buprenorphine, buquinolate, butacaine sulfate, butamisole hydrochloride, butorphanol tartrate, cambendazole, capromorelin, caramiphen edisylate, carbadox, carbomycin, carbon dioxide, carfentanil citrate, carnidazole, carprofen, castor oil, cefadroxil, cefovecin sodium, cefpodoxime proxetil, ceftiofur crystalline free acid, ceftiofur hydrochloride, ceftiofur sodium, cephalexin, cephapirin benzathine, cephapirin sodium, chloral hydrate, chloramine-t trihydrate, chloramphenicol, chloramphenicol palmitate, chlorhexidine acetate, chlorhexidine hydrochloride, chlorobutanol, chloroquine phosphate, chlorothiazide, chlorphenesin carbamate, chlortetracycline, chl ortetracycline bi sulfate, chlortetracycline calcium complex, chlortetracycline hydrochloride, chorionic gonadotropin, chymotrypsin, citric acid, clavulanate potassium, clenbuterol hydrochloride, clindamycin hydrochloride, clodronate, clomipramine hydrochloride, clopidol, cloprostenol sodium, clorsulon, clotrimazole, cloxacillin benzathine, cloxacillin sodium, colistimethate sodium, colloidal ferric oxide, copper naphthenate, corticotropin, coumaphos, cupric glycinate, cyclosporine, cyclosporine oral solution, cythioate, danofloxacin, decoquinate, deracoxib, deslorelin acetate, desoxycorticosterone pivalate, detomidine hydrochloride, dexamethasone, dexamethasone sodium phosphate, dexamethasone-21-isonicotinate, dexmedetomidine, dexmedetomidine hydrochloride, dextrose, diatrizoate meglumine, diatrizoate sodium, dibucaine hydrochloride, dichlorophene, dichlorvos, diclazuril, diclofenac sodium, dicloxacillin sodium monohydrate, diethylcarbamazine citrate, difloxacin hydrochloride, dihydrostreptomycin sulfate, dimethyl sulfoxide, dinoprost tromethamine, dipiperazine sulfate, diprenorphine hydrochloride, dirlotapide, dithiazanine iodide, domperidone, doramectin, doxapram hydrochloride, doxycycline hyclate, doxylamine succinate, droperidol, efrotomycin, embutramid, emodepside, enalapril maleate, enrofloxacin, eprinomectin, epsiprantel, erythromycin, erythromycin phosphate, erythromycin thiocyanate, estradiol, estradiol benzoate, estradiol valerate, estriol, ethopabate, ethylisobutrazine hydrochloride, etodolac, etorphine hydrochloride, famphur, febantel, fenbendazole, fenprostalene, fentanyl, fentanyl citrate, fenthion, firocoxib, florfenicol, flumethasone, flumethasone acetate, flunixin meglumine, fluocinolone acetonide, fluoxetine hydrochloride, fluprostenol sodium, fluralaner, follicle stimulating hormone, formalin, furazolidone, furosemide, gamithromycin, gelatin, gentamicin sulfate, gentamicin sulfate usp, gleptoferron, glycine, glycopyrrolate, gonadorelin acetate, gonadorelin diacetate tetrahydrate, gonadorelin hydrochloride, gonadotropin releasing factor—diphtheria toxoid conjugate, grapiprant, griseofulvin, guaifenesin, halofuginone hydrobromide, halothane, haloxon, helium, hemoglobin glutamer-200 (bovine), hetacillin potassium, hyaluronate sodium, hydrochloride, hydrochlorothiazide, hydrocortisone, hydrocortisone aceponate, hydrocortisone acetate, hydrogen peroxide, hygromycin b, imidacloprid, imidocarb dipropionate, iodinated casein, iodochlorhydroxyquin, iron dextran complex, isoflupredone acetate, isoflurane, isopropamide iodide, itraconazole, ivermectin, kanamycin sulfate, ketamine, ketamine hydrochloride, ketoprofen, laidlomycin propionate potassium, lasalocid, lasalocid sodium, levamisole, levamisole hydrochloride, levamisole phosphate, levamisole resinate, levothyroxine sodium, lidocaine, lincomycin, lincomycin hydrochloride, lincomycin hydrochloride monohydrate, liothyronine sodium, lufenuron, luprostiol, maduramicin ammonium, magnesium sulfate, marbofloxacin, maropitant, mebendazole, meclofenamic acid, medetomidine, medical air, megestrol acetate, melarsomine dihydrochloride, melatonin, melengestrol acetate, meloxicam, mepivacaine hydrochloride, methenamine mandelate, methocarbamol, methylprednisolone, methylprednisolone acetate, metoserpate hydrochloride, mibolerone, miconazole nitrate, milbemycin oxime, mometasone furoate, mometasone furoate anhydrous, mometasone furoate monohydrate, monensin, monensin sodium, monensin usp, morantel tartrate, moxidectin, mupirocin, myristyl-gamma- picolinium chloride, nalorphine hydrochloride, naltrexone hydrochloride, naproxen, narasin, n-butyl chloride, n-butylscopolammonium bromide, neomycin, neomycin palmitate, neomycin sulfate, neostigmine methylsulfate, nequinate, nf, nicarbazin, nitenpyram, nitrofurazone, nitrogen, nitromide, nitrous oxide, norgestomet, novobiocin, novobiocin sodium, nystatin, oclacitinib, oleate sodium, omeprazole, opafp-ghc2 rdna construct, orbifloxacin, orgotein, ormetoprim, oxfendazole, oxibendazole, oxygen, oxytetracycline, oxytetracycline (monoalkyl trimethyl ammonium salt), oxytetracycline dihydrate, oxytetracycline hydrochloride, oxytocin, pegbovigrastim, penicillin g benzathine, penicillin g potassium, penicillin g procaine, penicillin v potassium, pentazocine lactate, pentobarbital, pentobarbital sodium, pergolide mesylate, phenothiazine, phenylbutazone, phenylpropanolamine hydrochloride, phenytoin sodium, phosmet, pimobendan, piperazine citrate, piperazine dihydrochloride, piperazine hydrochloride, piperazine monohydrochloride, piperazine phosphate, piperazine-carbon disulfide complex, pirlimycin hydrochloride, pituitary luteinizing hormone, poloxalene, polymyxin b sulfate, polyoxyethylene 23 lauryl ether, polysulfated glycosaminoglycan, ponazuril, porcine insulin zinc, porcine pituitary-derived follicle stimulating hormone, posaconazole, potassium, potassium citrate, potassium phosphate, pradofloxacin, pralidoxime chloride, praziquantel, prednisolone, prednisolone acetate, prednisolone sodium phosphate, prednisolone sodium succinate, prednisolone tertiary butylacetate, prednisone, primidone, prochlorperazine dimaleate, prochlorperazine edisylate, prochlorperazine maleate, progesterone, promazine hydrochloride, proparacaine hydrochloride, propiopromazine hydrochloride, propofol, prostalene, pyrantel pamoate, pyrantel tartrate, pyrilamine maleate, pyrimethamine, rabacfosadine, ractopamine hydrochloride, robenacoxib, robenidine hydrochloride, romifidine hydrochloride, roxarsone, salinomycin sodium, sarolaner, secobarbital sodium, selamectin, selegiline hydrochloride, selenium disulfide, semduramicin sodium, semduramicin sodium biomass, serum gonadotropin, sevoflurane, silver sulfadiazine, sodium chloride, sodium selenite, sodium sulfachloropyrazine monohydrate, sodium sulfachlorpyridazine, sodium sulfamethazine, spectinomycin, spectinomycin dihydrochloride pentahydrate, spectinomycin hydrochloride pentahydrate, spectinomycin sulfate tetrahydrate, spinosad, stanozolol, streptomycin sulfate, sulfabromomethazine sodium, sulfachlorpyridazine, sulfadiazine, sulfadiazine sodium, sulfadimethoxine, sulfaethoxypyridazine, sulfamerazine, sulfamethazine, sulfamethazine bisulfate, sulfamethizole, sulfanitran, sulfaquinoxaline, sulfaquinoxaline sodium, sulfisoxazole, sulfomyxin, tepoxalin, terbinafine, testosterone propionate, tetracaine hydrochloride, tetracycline, tetracycline hydrochloride, tetracycline phosphate, thenium closylate, thiabendazole, thialbarbitone sodium, thiamylal sodium, thiopental sodium, thiostrepton, thyroid stimulating hormone, tiamulin, tiamulin hydrogen fumarate, ticarcillin disodium, tildipirosin, tiletamine hydrochloride, tilmicosin phosphate, tiludronate disodium, tioxidazole, toceranib phosphate, tolazoline hydrochloride, tolnaftate, toluene, trenbolone acetate, triamcinolone acetonide, tricaine methanesulfonate, trichlorfon, trichlormethiazide, triflupromazine hydrochloride, trilostane, trimeprazine tartrate, trimethoprim, tripelennamine hydrochloride, triptorelin acetate, trypsin, tulathromycin, tylosin, tylosin phosphate, tylosin tartrate, tylvalosin, tylvalosin tartrate, clotrimazole, virginiamycin, vitamin E, xylazine, xylazine hydrochloride, yohimbine hydrochloride, zeranol, zilpaterol, zilpaterol hydrochloride, zinc gluconate, zoalene, and zolazepam hydrochloride.

The compositions of various embodiments may include any of the active agents identified above or combinations thereof in an effective amount. For example, such compositions for topical administration such as, but not limited to, a solutions, powders, fluid emulsions, fluid suspensions, solid, semi-solids, ointments, pastes, creams, gels and jellies, foams, or aerosol may include about 0.01 wt. % to about 50 wt. % active agent or in some embodiments, about 0.1 wt. % to about 25 wt. % active agent, or any amount encompassed by these example ranges. The person of ordinary skill in the art can determine the dosage based on known factors associated with the active agents identified above. In some embodiments, the therapeutically effective amount may be about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 mg, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values.

Particular examples of compositions encompassed by the invention include compositions containing about 0.1 wt. % to about 2.0 wt. % decoy molecule having an average molecular weight of about 2,000 Da to about 60,000, and active agent such as salicylate, lidocaine, sunblock, retinol, bimatoprost, various steroids, and active agents of similar size and combinations thereof. Other examples of compositions encompassed by the invention include compositions containing about 0.5 wt. % to about 5.0 wt. % decoy molecule having an average molecular weight of about 2,000 Da to about 60,000, and one or more active agent such as antibiotics, antifungal agents, biologics, antibodies, macromolecule active agents, peptide-based therapeutics, and active agents of similar size and combinations thereof.

In some embodiments, the compositions described above may further include one or more pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, N.Y. (1980) for guidance in determining the amount of such components in the compositions and formulations of embodiments.

In some embodiments, the compositions described above may be formulated as a liquid. Liquid dosage forms for topical administration may include diluents such as, for example, alcohols, glycols, oils, water, and the like. Such compositions may also include wetting agents or emulsifiers. In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanoilin, wool alcohol, stearyl alcohol, etc.) or silicones. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

In particular embodiments, the compositions described above can be formulated as aerosols in which the composition is dissolved in a propellant such as dichlorodifluoromethane, trichlorofluoromethane, dichlorotetrafluoroethane, carbon dioxide, or other suitable gas, and a co-solvent such ethanol, acetone, hexadecyl alcohol, and the like and combinations thereof.

In certain embodiments, the compositions of various embodiments may be formulated for improving appearance of skin and may additionally include additives such as vitamins, cosmetic peptides, oil control agents, and other skin care agents.

Vitamins include, for example, vitamin D, vitamin K, vitamin B (including niacinamide, nicotinic acid, C₁₋₁₈ nicotinic acid esters, and nicotinyl alcohol; B6 compounds, such as pyroxidine; and B5 compounds, such as panthenol, or “pro-B5”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

Peptides include di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (MATRIXYL®) palmitoyl-glycine-glutamine-proline-arginine (RIGIN®), these three being available from Sederma, France, and Cu-histidine-glycine-glycine (Cu-HGG, also known as IAMIN®), and naturally occurring and synthesized derivatives thereof, and combinations thereof In various embodiments, the compositions may include about 1×10⁻⁷ wt. % to about 20 wt. %, about 1×10⁻⁶ wt. % to about 10 wt. %, and about 1×10⁻⁵ wt. % to about 5 wt. %, or any individual concentration or range of each peptide contained in the composition.

Oil control agents include compounds useful for regulating the production of skin oil, or sebum, and for improving the appearance of oily skin. Examples of oil control agents include, for example, salicylic acid, dehydroacetic acid, benzoyl peroxide, vitamin B3 (for example, niacinamide), and the like, their isomers, esters, salts and derivatives, and mixtures thereof. The compositions of such embodiments may include about 0.0001 wt. % to about 15 wt. %, about 0.01 wt. % to about 10 wt. %, about 0.1 wt. % to about 5 wt. %, and about 0.2 wt. % to about 2 wt. %, or any individual concentration or range of each oil control agent contained in the composition.

Other skin care agents include retinol, steroids, sunblock, salicylate, minocycline, antifungals, peptides, antibodies, lidocaine, and the like and combinations thereof. In some embodiments, other skin care agents include N-acyl amino acid compounds includinf, for example, N-acyl phenylalanine, N-acyl tyrosine, and the like, their isomers, including their D and L isomers, salts, derivatives, and mixtures thereof. An example of a suitable N-acyl amino acid is N-undecylenoyl-L-phenylalanine is commercially available under the tradename SEPIWHITE®. Further skin care agents are disclosed in US Publication No. 2007/0020220A1, wherein the components/ingredients are incorporated herein by reference in their entirety.

The compositions of embodiments described above may enhance the strength of known topical active agent thereby reducing the necessary dosage required to achieve a therapeutically effective amount. For example, in some embodiments, the strength of a composition containing an active agent and a decoy molecule may be about equal to about 80% or 90% greater than the active agent delivered in a standard topical formulation. In other embodiments, the strength of a composition containing an active agent and a decoy molecule may be about equal to about 75% greater, about 1.0% to about 80% greater, about 1.0% to about 75% greater, about 1.0% to about 50% greater, about 1.0% to about 25% greater, about 2.0% to about 80% greater, about 2.0% to about 75% greater, about 2.0% to about 50% greater, about 2.0% to about 25% greater, about 5.0% to about 50% greater, about 5.0% to about 25% greater, or any range or individual strength encompassed by these example ranges. Thus, the compositions described herein may provide therapeutic equivalence of known topically administered active agents with that an administered dose that is equal to or at least about 75% less than a standard dose, equal to or about 50% less than a standard dose, equal to or about 25% less than a standard dose, equal to or about 10% less than a standard dose, about 1.0% to about 75% less than a standard dose, about 1.0% to about 50% less than a standard dose, about 1.0% to about 25% less than a standard dose, about 1.0% to about 10% less than a standard dose, about 2.0% to about 75% less than a standard dose, about 2.0% to about 50% less than a standard dose, about 2.0% to about 25% less than a standard dose, about 2.0% to about 10% less than a standard dose, or any range or individual value encompassed by these example ranges.

A wide variety of methods may be used for preparing the formulations described above. Broadly speaking, the formulations may be prepared by combining together the components of the formulation, as described herein, at a temperature and for a time sufficient to provide a pharmaceutically acceptable composition. For example, in some embodiments, the compositions components of the compositions may be dissolved, suspended, dispersed or otherwise mixed in a selected carrier or vehicle, at an effective concentration such that the condition to be treated is relieved or ameliorated.

Further embodiments are directed to devices including the compositions described above. For example, such compositions and formulations can be coated on bandages, mixed with bioadhesives, or included in wound dressings.

Additional embodiments include methods for delivering an active agent. Some embodiments may include the step of co-administering an active agent and a decoy molecule to a surface tissue. For example, such methods may include the step of applying a composition or formulation such as those described above including an active agent and a decoy molecule to a surface tissue of a of a subject. In other embodiments, the decoy molecule may be applied to the surface tissue before topical administration of the active agent. For example, a wipe containing a composition include one or more decoy molecules may be used for applying a decoy molecule to surface tissue followed by a step of topically administering an active agent to the surface tissue. In yet other embodiments, the active agent may be applied to a surface tissue followed by applying a decoy molecule to the surface tissue.

As indicated above, a “surface tissue” includes any surface tissue such as, but not limited to, skin, mucosa, eyes, ears, inside the nose, inside the mouth, lips, urethral openings, vagina, anus, tongue, frenulum of tongue, hair, teeth, and the like. The methods of such embodiments may include a variety of additional steps including, for example, cleaning the surface tissue at the site of applying and the like. In such embodiments, the composition can be applied to the surface tissue one or more times each day, and applying can be carried out for a period of at least 1 month, 2 months, 3 months, 4 months, 6 months, 8 months or 12 months.

The methods of such embodiments can be used for treating nearly any condition. For example, the methods of embodiments can be used for treatment of a variety of skin conditions including acne, local pain relief, local fungal or bacterial infections, skin cancer, abscesses, cellulitis, and the like. In other embodiments, the methods may be used to for administration of various cosmetic therapies for improving, for example, skin thickness, elasticity, resiliency, smoothness, tone, texture, brightness, clarity, contour, firmness, tautness, suppleness, discoloration, skin lesions, and the like and combinations thereof. The methods of further embodiments can be used for enhancing the color or strength of, for example, hair or teeth. In still other embodiments, the methods of the invention can be used for administering active agents for treating numerous systemic conditions in which transdermal delivery of the active agent is preferred, for example, chronic pain relief, cancer, motion sickness, chronic illnesses, and the like and combinations thereof.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples.

### Example 1

### Hylauronic Acid and Biomimetic Peptides

Compositions containing of a mixture of peptides that promote hair growth were prepared. The peptides, sold under the tradename Renokin®, include decapeptide-10, oligopeptide-54 (CG-Nokkin), decapeptide -18, acetyl decapeptide-3, and oligopeptide-42. The peptide compositions were prepared by mixing the peptides in saline along with a decoy molecule of hyaluronic acid with a molecular weight of 10,000 Daltons, 20,000 Daltons, 40,000 Daltons, 60,000 Daltons, or 100,000 Daltons. Control formulations were comprised of the peptides alone and of saline alone.

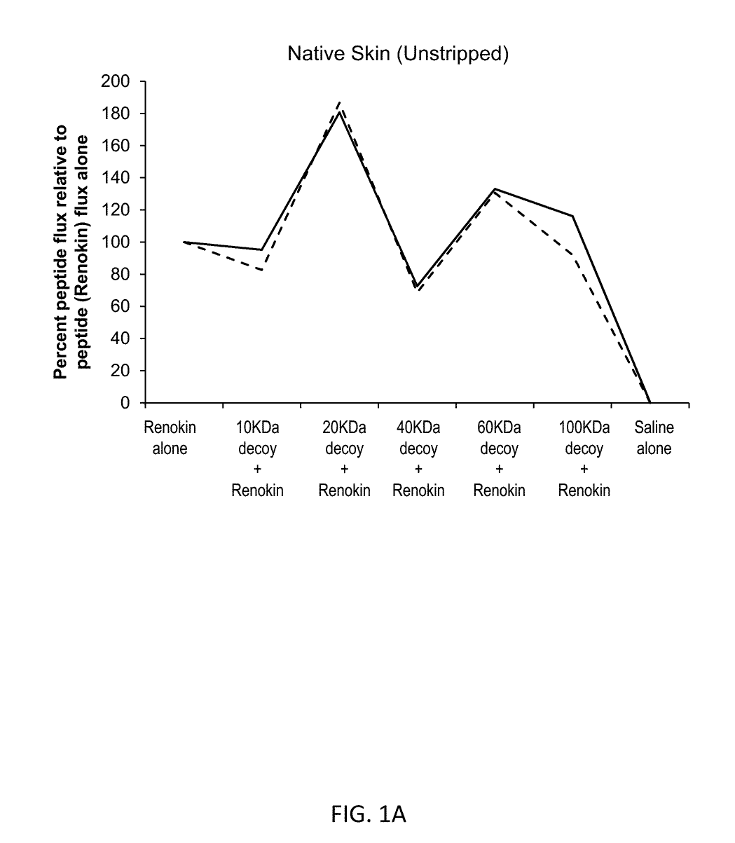

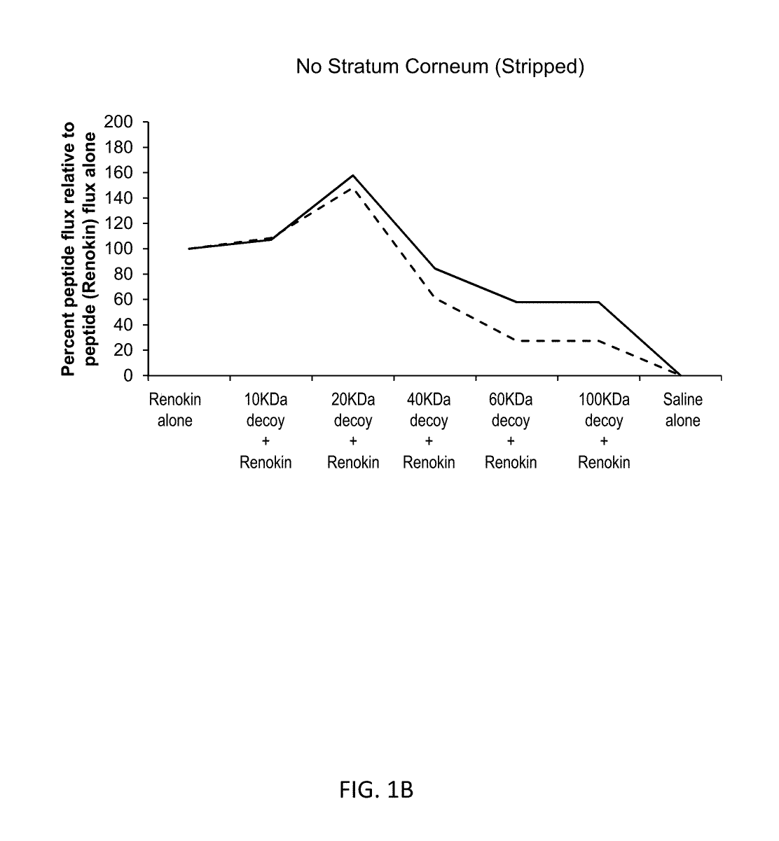

FIG. 1A shows the results for the studies conducted using skin with intact stratum corneum. This demonstrates partially passive binding, receptor mediated enhancement patterns are present and bimodal specific enhancement is present; nonspecific water enhancement would increase as size increases so the enhanced penetration effect is specific. Addition of progressively larger molecular weights reverses the benefit even with dead skin present.

FIG. 1B shows the results for the studies conducted using skin with stratum corneum stripped off using the tape stripping method. This demonstrates active binding, receptor mediated enhancement pattern across viable skin layers without stratum corneum (i.e. without water enhancement effect at all) and specific enhancement present based on MW; larger MW not only abolishes enhancement but retards penetration across cells in the viable skin layers which present the barrier to deep epidermal and dermal penetration.

The percent of peptide flux relative to flux of peptide from the composition of peptide alone is shown for each of the test compositions. Each composition was tested twice, the first study indicated by the solid line, and the second study by the dashed line. Hyaluronic acid with a molecular weight up to 300,000 Da is known to be able to penetrate skin (Essendoubi, M, et al, Skin Res. and Tech, 22:55-62 (2016)). The data in FIGS. 1A-1B show that delivery of the peptides using a hyaluronic acid molecule of less than about 40,000 Da is via a delivery path different than that for a hyaluronic acid molecule of greater than 40,000 Da, and that neither delivery path is purely related to a hydration effect. When stratum corneum is present on the skin (FIG. 1A), a peak in peptide delivery is observed from compositions with a hyaluronic acid of 20,000 Da and 60,000 Da. When stratum corneum is stripped from the skin (FIG. 1B), the peak achieved using a 60,000 Da hyaluronic acid decoy molecule is not observed, demonstrating that peptide delivery is not due to a hydration effect alone since enhancement of skin penetration due to hydration of the skin would increase with increasing decoy molecular weight. Further, since it is known that 100,000 Da hyaluronic acid penetrates the stratum corneum (Essendoubi, 2016), if the delivery observed from the present compositions was due to hydration it would be expected to observe peptide delivery from compositions with a 100,000 Da hyaluronic acid decoy molecule across skin with and without stratum corneum. FIG. 1B shows that the composition with 100,000 Da hyaluronic acid decoy molecule provided less delivery of peptide than did compositions with molecular weight hyaluronic acid. The compositions with a decoy molecule of 40,000 Da and less enhanced delivery of the peptides, relative to delivery from compositions with no decoy molecule (FIG. 1A).

### Example 2

### Hylauronic Acid and Salicylate

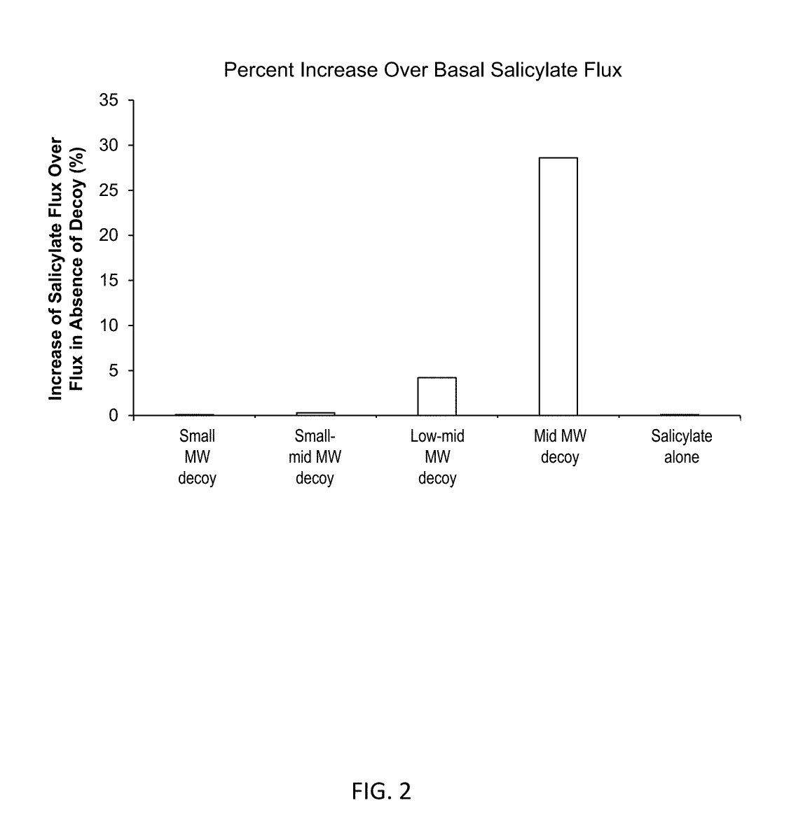

Compositions were prepared containing 1% salicylate and 1% of decoy molecule of hyaluronic acid with four molecular weights: small (5,000 Da to 10,000 Da), small to mid (10,000 Da to 20,000 Da), low to mid (20,000 Da to 30,000 Da), and mid (30,000 Da to 40,000 Da). A control formulation containing salicylate alone was also prepared. The compositions were placed in Franz diffusion cells with skin separating the compartments of the diffusion cell. The concentration of salicylate in the receiver side of the diffusion cells was measured after a fixed time and the results are shown in FIG. 2.

The composition with the 10,000 Da to 20,000 Da decoy of hyaluronic acid achieved a 27% higher flux of salicylate compared to the flux of salicylate from the composition of salicylate alone. The 20,000 Da to 30,000 Da decoy molecule increased salicylate skin flux about 5% compared to the flux of salicylate from the composition of salicylate alone.

### Example 3

### Hylauronic Acid and a Steriod

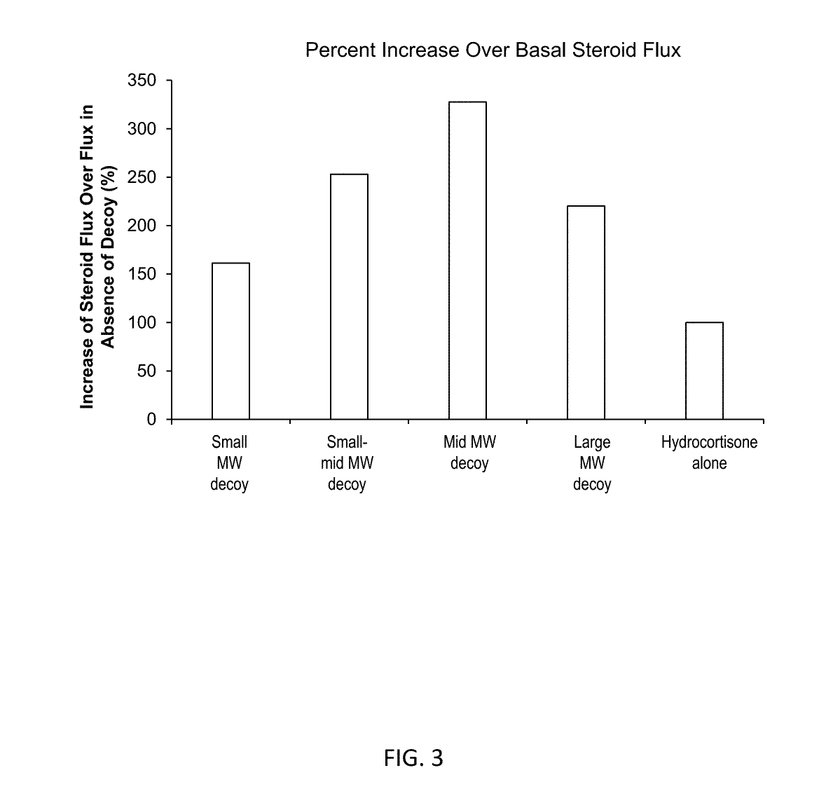

Compositions were prepared containing 1% hydrocortisone and 1% of decoy molecule of hyaluronic acid with four molecular weights: small (5,000 Da to 10,000 Da), small to mid (10,000 Da to 20,000 Da), low to mid (20,000 Da to 30,000 Da), and mid (30,000 Da to 40,000 Da). A control formulation containing hydrocortisone alone was also prepared. The compositions were placed in Franz diffusion cells with skin separating the compartments of the diffusion cell. The concentration of salicylate in the receiver side of the diffusion cells was measured after a fixed time and the results are shown in FIG. 3.

The compositions with the hyaluronic acid decoy molecules increased delivery of hydrocortisone across the skin, with the mid-sized decoy of 20,000 Da to 30,000 Da giving a 325% increase in hydrocortisone flux compared to flux of hydrocortisone from a composition lacking the decoy molecule. The small-to-mid-sized decoy molecule with a molecular weight of about 10,000 Da to 20,000 Da increased salicylate skin flux about 250% compared to flux of hydrocortisone from a composition with no decoy molecule.

### Example 4

### Elastin and Lidocaine

Delivery of lidocaine across skin was evaluated using compositions containing an elastin decoy molecule. Compositions containing of 1 wt. % lidocaine and 0.5 wt % of a decoy of elastin in saline were prepared with three molecular weights: very very small (2,000 Da to 5,000 Da), very small (5,000 Da to 10,000 Da), and small (10,000 Da to 20,000 Da).

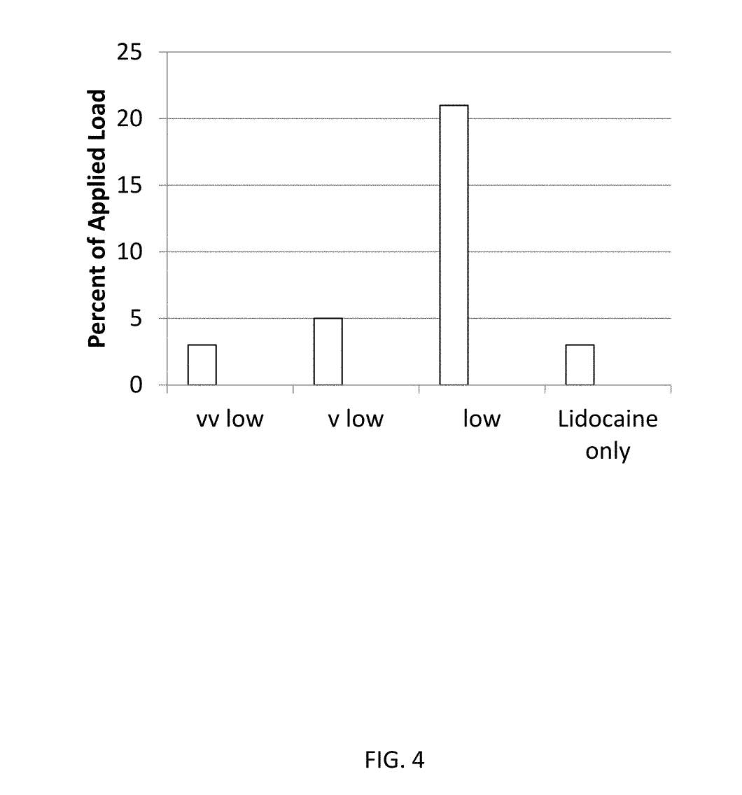

Viable porcine skin was obtained and used to produce mid-dermal grafts (0.045-0.055 units). The grafts were positioned in transcutaneous flux devices. Flow in the devices was maintained at the lowest setting and all receptor fluid was collected for each replicate (n=8 for each of the test formulation and the control formulation). Flux was continued for 12-20 hours with samples applied and left on donor skin surfaces. The skin for each cell (each chamber) was washed, then homogenized. The clarified homogenate solution and the flow through samples were assayed for lidocaine content using spectroscopy. After a 12-20 hour permeation period, the concentration of lidocaine in the skin was determined. The results are shown in FIG. 4 as the percent of applied lidocaine.

The lidocaine formulation with no decoy molecule achieved 3% penetration. Addition of an elastin decoy molecule having a small molecular weight (10,000 Da to 20,000 Da) enhanced skin penetration by about 7 fold (significant improvement in penetration, p=0.0001).

### Example 5

### Hylauronic Acid and Minocycline

Oral minocycline HCl is highly effective but limited by ototoxicity and emerging resistance. Majority of physicians would use topical minocycline versus oral. However, topical application is currently less effective than oral because minocycline does not effectively cross skin. As a result, higher concentrations must be used and these discolor skin and textiles.

Delivery of minocycline into porcine skin in vitro was measured and compared to delivery of minocycline from a composition of minocycline in saline (i.e, with no decoy molecule). Compositions were prepared containing of 1 wt. % minocycline and 1% of decoy molecule of hyaluronic acid with three molecular weights: 10,000 Da mean, 20,000 Da mean, and 30,000 Da mean. A control formulation containing 1 wt % minocycline in saline was also prepared

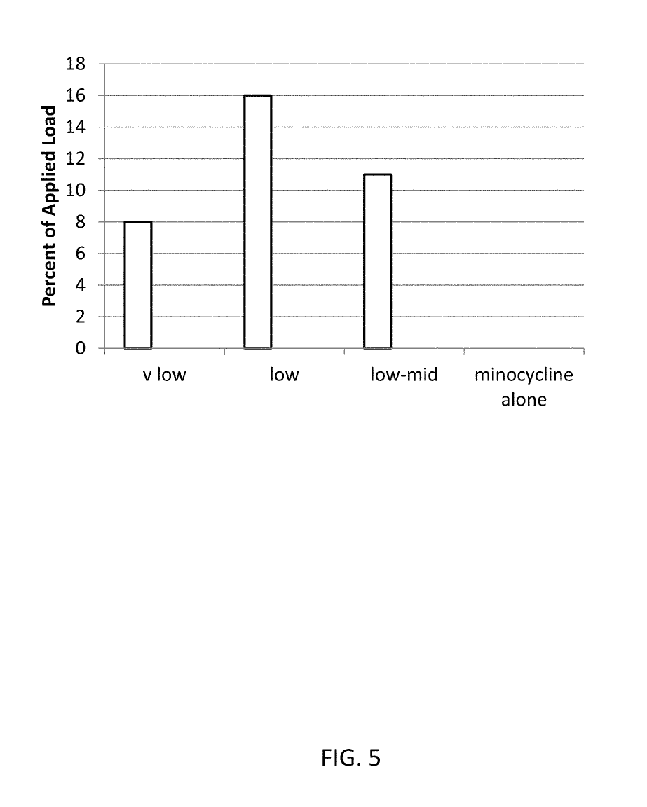

FIG. 5 shows the results of the study, where the amount of minocycline in tissue, μg minocycline/g tissue, delivered into the porcine skin grafts from the topical formulations of minocycline and sodium hyaluronate is shown by the bars with dashed fill and from the topical formulation of minocycline without sodium hyaluronate by the bar with open fill. Although minocycline had low native penetration, the polysaccharide-based decoys enhanced penetration significantly (p=0.0004). These results confirm that a decoy-mediated strategy can afford high penetration of a topical minocycline. A decoy molecule with a low molecular weight increased the very low basal penetration of minocycline to levels that can achieve higher tissue concentrations than oral while avoiding discoloration and systemic side effects. A topical composition containing minocycline with a decoy molecule can be used for treating or ameloriating skin structure infections or disorders, such as cellulitis.

### Example 6

Compositions For Protection of Skin from UVA/UVB Rays

Current chemical agents used for sunblock have poor compliance due to thick bases, incompatibility with cosmetics, and short duration. By enhancing function of existing agents, it becomes possible to develop a more effective sunblock, a sunblock which is resistant to rubbing off, and/or a more desirable formulation feel and use with other products (to induce better compliance).

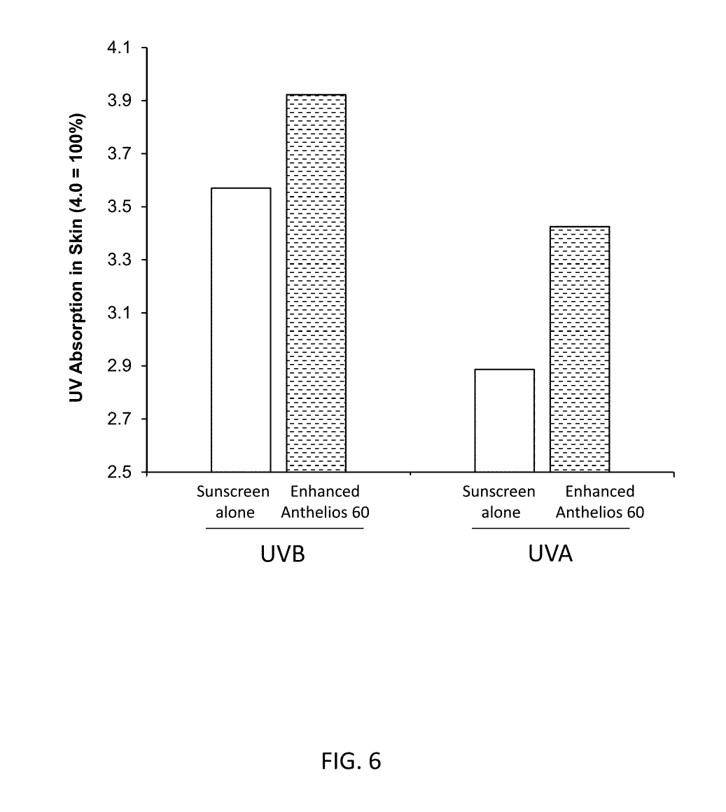

In this study, compositions for protection of skin from UV-A and/or UV-B exposure were prepared and tested. Groups include A) Laroche Posay Anthelios 60 Sunblock spiked with 1:10 saline (n=10 replicates), or B) Laroche Posay Anthelios 60 Sunblock spiked with 1:10 1% sodium hyaluronate of molecular weight 10,000 (“enhanced Anthelios 60”) in saline (n=10 replicates) in donor cells. Flow was maintained at the lowest setting and all receptor fluid was collected for each replicate. Flux was continued for 12-20 hours with samples applied and left on donor surfaces. The skin for each cell (each chamber) was washed, then then punch biopsied, placed into 96 well plates and employed in full range UV spectra. UV absorbance per group was determined by wavelength for each group and UVA and UVB values determined from the appropriate wavelengths. Results are shown in FIGS. 6, 7A-7B, and 8.

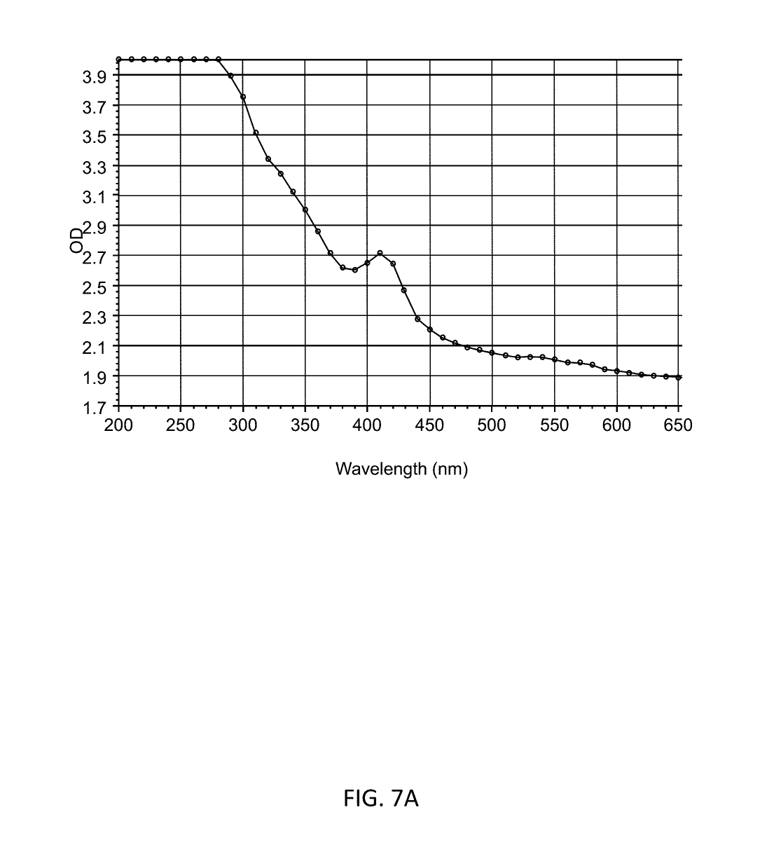

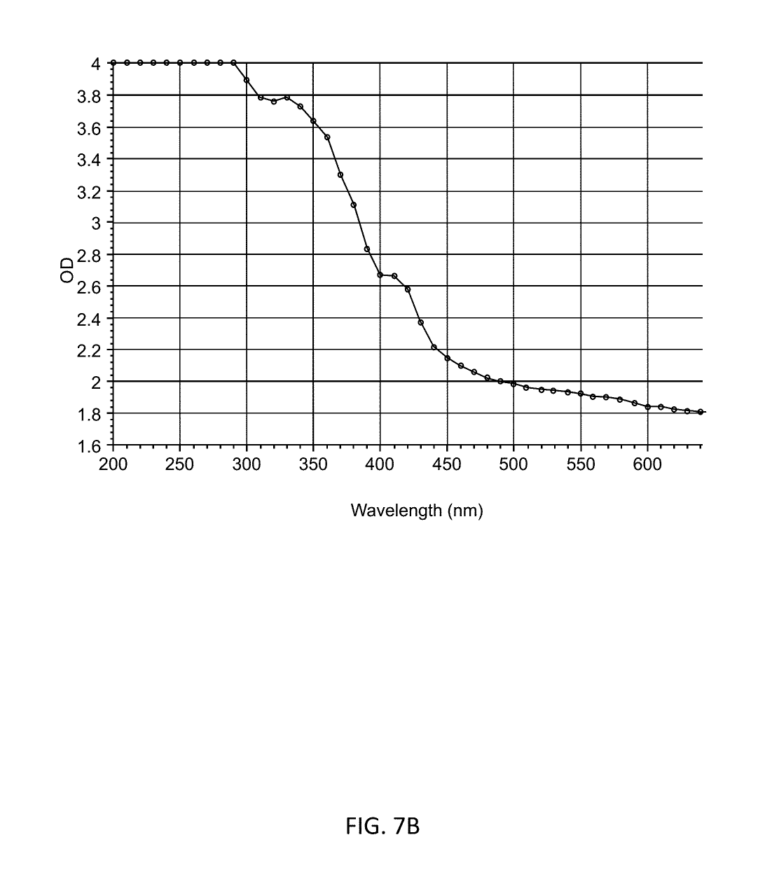

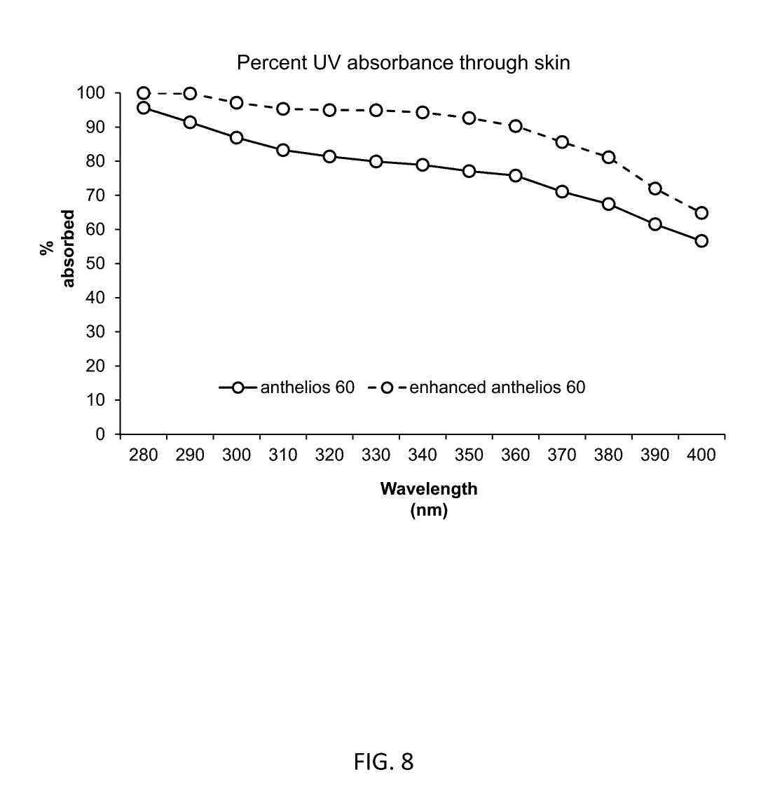

Addition of an enhancer which has no UV absorbance itself, increased the performance of a commercially available mix of UV blocking agents statistically significantly across both UVA (P=0.001) and UVB (P=0.001) as depicted in FIG. 6. Individual wavelength results by group are shown in FIG. 8 and one representative spectrum from each group is presented as FIGS. 7A and 7B.

The compositions with and without decoy molecule were tested to determine UV absorption in skin. FIG. 6 is a bar graph (4.0 corresponds to 100%) showing the absorption of UVA and UVB in skin, where the bars with dashed fill correspond with the sunscreen compositions with a decoy molecule and the solid white bars are sunscreen alone.

FIGS. 7A-7B are graphs of UV absorption as a function of wavelength, in nm, for commercially available sunscreen (Anthelios 60) (FIG. 7A) and for the commercially available sunscreen (Anthelios 60) with a decoy molecule, enhanced Anthelios 60 (FIG. 7B).

FIG. 8 is a graph showing the percent UV absorbance through skin as a function of wavelength, in nm, for commercially available sunscreen (Anthelios 60) (solid line) and for the commercially available sunscreen (Anthelios 60) with a decoy molecule, enhanced Anthelios 60 (dashed line).

### Example 7

### Hyaluronic Acid and Gabapentin

Delivery of gabapentin with hyaluronic acid into skin in vitro was measured using porcine skin grafts, and compared to delivery of gabapentin from a composition of gabapentin in saline (with no decoy molecule). Groups consisted of A) 1% gabapentin in saline (n=8 replicates), B) 1% gabapentin plus 1% sodium hyaluronate decoy of 3,000 Da in saline (n=8 replicates) or C) saline alone (n=8 replicates) in donor cells.

Viable porcine skin was processed to produce mid-dermal grafts (0.045-0.055 units) and the grafts were positioned in transcutaneous flux devices. Flow in the devices was maintained at the lowest setting and all receptor fluid was collected for each replicate (n=8 for each of the test formulation and control formulations). Flux was continued for 12-20 hours with samples applied and left on donor skin surfaces. The skin for each cell (each chamber) was washed, then employed in an assay of gabapentin content within the skin sample using a UPLC-mass spectrometer method. Briefly, tissues were incubated overnight in 0.5 mL of 50% acetonitrile in deionized water at 55° C. with agitation. Calibration standards and tissue extraction solvent samples were diluted 10× in deionized water before analysis. Diluted standards and samples were analyzed at 1 μL injection volumes. Concentrations were reported as μg/g of gabapentin in tissue.

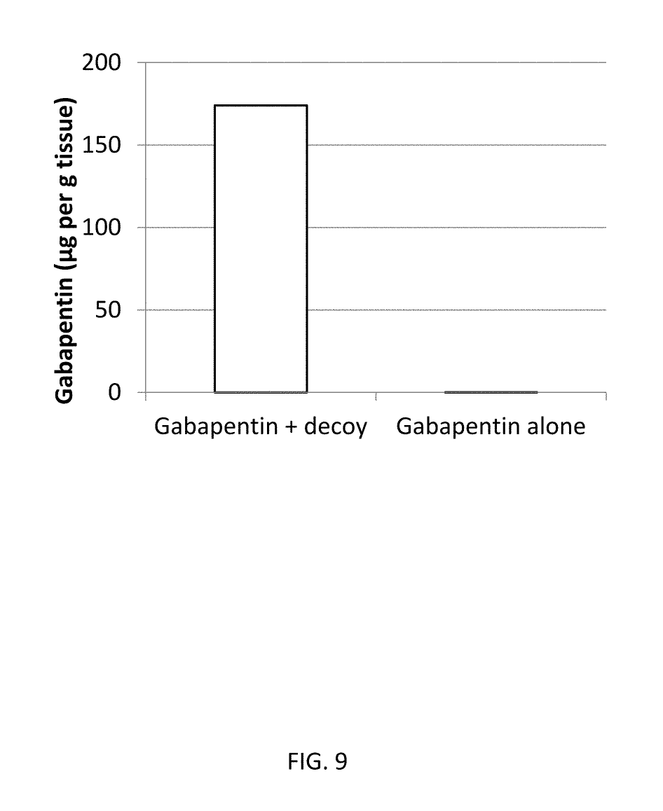

FIG. 9 shows the results of the study, where the amount of gabapentin in tissue, μg gabapentin/g tissue, delivered into the porcine skin grafts from the topical formulation of gabapentin and sodium hyaluronate and the formulation of gabapentin without sodium hyaluronate are shown. Gabapentin alone did not yield significant penetration above saline (p=0.99) but gabapentin in the presence of the decoy achieved significant penetration versus both saline (p=0.018) and gabapentin alone (p=0.013). Specifically, gabapentin alone yielded tissue levels of 0.09 μg of gabapentin per gram of tissue while gabapentin with the addition of a decoy molecule yielded tissue levels of 174.01 μg of gabapentin per gram of tissue. Thus, the addition of a decoy molecule yielded a 1,900 fold increase in delivery of the agent to the skin, and a statistically significant increased penetration of gabapentin topically.

### Example 8

Hyaluronic Acid and Palmitoyl-lysine-threonine-threonine-lysine-serine

A topical composition containing a cosmetic agent, palmitoyl-lysine-threonine-threonine-lysine-serine (pal-KTTKS) and sodium hyaluronate (3,000 Da) as a decoy molecule were prepared. Groups consisted of A) 1% Pal-KTTKS spiked into Olay ProX (n=8 replicates), or B) 1% Pal-KTTKS spiked into Olay ProX plus 1% sodium hyaluronate decoy of 3,000 Da in saline (n=8 replicates).

Viable porcine skin was processed to produce mid-dermal grafts (0.045-0.055 units) and the grafts were positioned in transcutaneous flux devices. Flow in the devices was maintained at the lowest setting and all receptor fluid was collected for each replicate. Flux was continued for 12-20 hours with samples applied and left on donor skin surfaces. The skin for each cell (each chamber) was washed, then homogenized. The clarified homogenate solution and the flow through samples were then employed in an assay of pal-KTTKS content within the skin sample using a UPLC-mass spectrometer method.

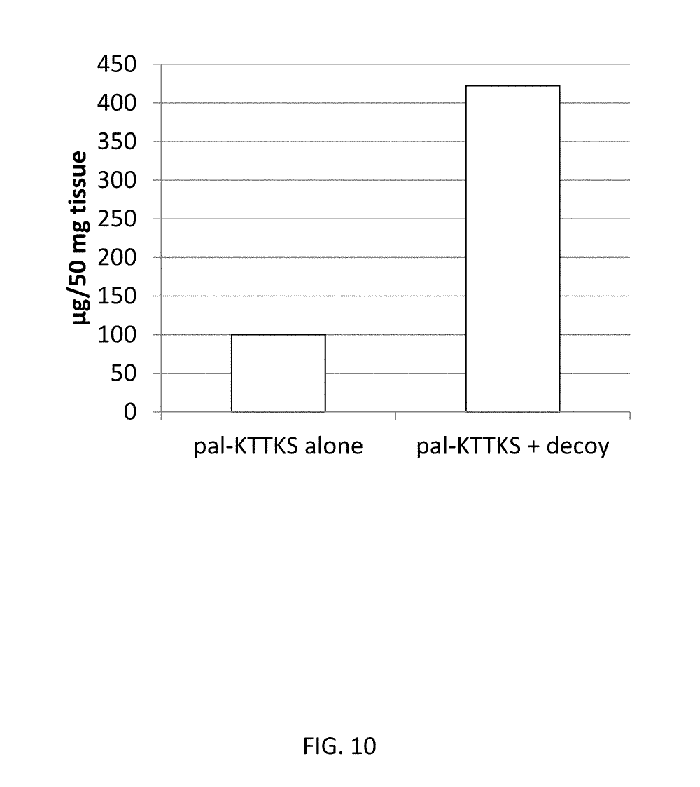

FIG. 10 shows the results of the study, where the amount of pal-KTTKS in the tissue (μg pal-KTTKS/50 mg tissue) delivered from the topical formulation of pal-KTTKS and sodium hyaluronate decoy and the topical formulation of pal-KTTKS without sodium hyaluronate are indicated. A formulation of pal-KTTKS alone (with no decoy molecule) after the 12-20 hour permeation period yielded about 100 μg pal-KTTKS/50 mg tissue. Addition of a decoy molecule improved permeation of the agent into the skin, with nearly 450 μg pal-KTTKS/50 mg tissue. Thus, the addition of a decoy molecule to the topical composition yielded a nearly 422% increased flux without optimization (P<0.01) in delivery of the agent to the skin. Thus, without any additional formulation change, a polysaccharide decoy provided substantial and significant enhancement in penetration of the most widely recognized peptidyl skincare active.

### Example 9

Ocular Delivery of FITC-Dextran from Compositions Containing a Decoy

Intact fresh, viable porcine eyes were obtained with full orbit uninjured. Eyes were bathed to midline (lens down) in treatment solution overnight while suspended superiorly via ligature of the optic nerve. Compositions were prepared as follows: A) 5,000 Da FITC-dextran in saline (n=2 replicates), B) 5,000 Da FITC-dextran in 1% sodium hyaluronate of 3,000 Da in saline (n=2 replicates), C) 5,000 Da FTIC-dextran in 0.5% short elastin in saline (n=2 replicates), and D) saline alone.

Eyes were thoroughly washed 5 times in saline then snap frozen and analyzed with a reflectance confocal imaging system (Vivascope 1500) to noninvasively image and visualize penetration of the FITC-dextran. The confocal microscopy showed that though almost no gross signal was present within the lens, both polysaccharide and peptidyl decoy molecules provided for visible penetration of the FITC-dextran marker (drug model) to the aqueous humor, including the anterior and posterior chamber and ciliary body; to the structural elements including zonule and sclera; and to the vitreous humor including bathing the retina. Saline controls showed no granular fluorescence and no drug (marker) penetration since no FITC-dextran was present.

This experiment confirms that a 5,000 Da drug marker penetrated into the eye when combined with both classes of decoy. A similar experiment using both dextran and antibody markers at 150,000 MW confirmed penetration with both classes of decoys; though differing magnitudes of flux for 150,000 versus 5,000 MW, both exhibited penetration when applied topically to intact eyes.

### Example 10

Delivery of FITC-Dextran to the Nail Unit from Compositions Containing a Decoy

A mixture of 1% 5,000 Da FITC-dextran and 1% 10,000 Da sodium hyaluronate was added to commercially available nail base at a 1:10 dilution. The material was applied to a toenail and allowed to stand for 3 hours. Confocal imaging was employed as before to view penetration of FITC-dextran into the nail plate. Images were acquired at 7 micron steps.

Very high levels of signal were present on the nail surface as expected. High levels of the 5,000 Da FITC-dextran conjugate were observed penetrating into the deepest layers of the nail plate as visualized by granular fluorescence patterns. Most antifungal and nutritional components of interest for the nail could thus be delivered through addition of a small decoy fragment.

### Example 11

Mucosal Delivery of Salicylate from Compositions Containing a Decoy Molecule

The compositions are contemplated for delivery of an agent to mucosal tissue, and a study was conducted using viable porcine buccal tissue to evaluate mucosal penetration of salicylate from compositions with an elastin decoy molecule. The following compositions were prepared for testing: A) 1% sodium salicylate in saline (n=4 replicates), or B) 1% sodium salicylate plus 0.5% short elastin fragment decoy (decoy) in saline (n=4 replicates).

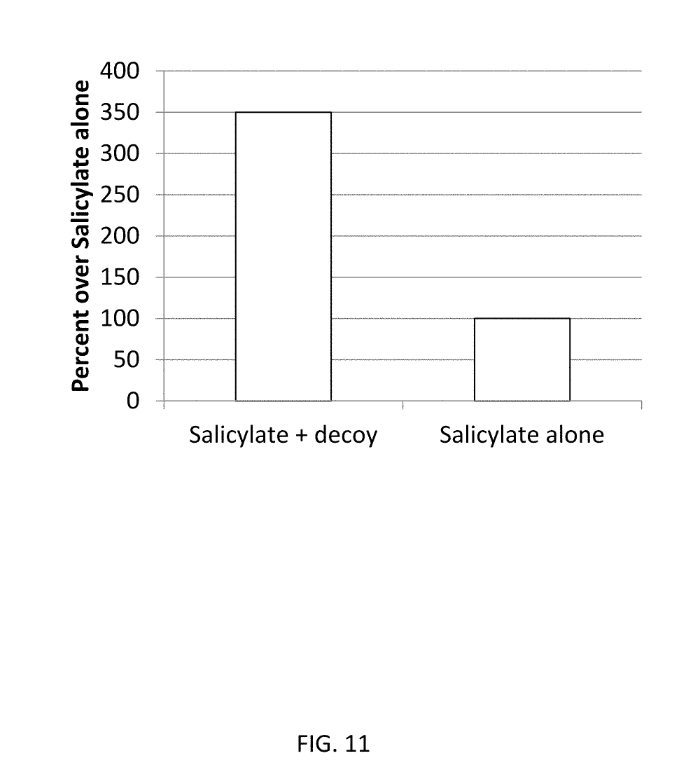

Viable porcine buccal tissue was obtained and grafts were produced. The grafts were placed in transcutaneous flux devices to measure mucosal penetration. Flow in the devices was maintained at the lowest setting and all receptor fluid was collected for each replicate (n=8 for each of the test formulation and control formulations). Flux was continued for 12-20 hours with samples applied and left on donor mucosal tissues. After the 12-20 hour test period tissue from each cell was washed, then homogenized. The clarified homogenate solution and the flow through samples were then employed in an assay of salicylate content via absorbance. The skin penetration of salicylate from a composition with an elastin decoy and from a composition with no decoy is shown in FIG. 11.

These results show that the addition of a decoy molecule to the composition achieved a 350% increase in mucosal penetration of salicylate.

### Example 12

Delivery of Antibody from Compositions Containing a Decoy Molecule

Compositions were prepared consisting of: A) 25 μl of an alkaline phosphatase conjugated IgG antibody in saline (n=8 replicates), B) 25 μl of an alkaline phosphatase conjugated IgG antibody plus 1% sodium hyaluronate of 3,000 Da in saline (n=8 replicates), C) 25 μl of an alkaline phosphatase conjugated IgG antibody plus 1% sodium hyaluronate of 5,000 Da in saline (n=8 replicates), or D) 25 μl of an alkaline phosphatase conjugated IgG antibody plus 1% sodium hyaluronate of 10,000 Da in saline (n=8 replicates) in donor cells.

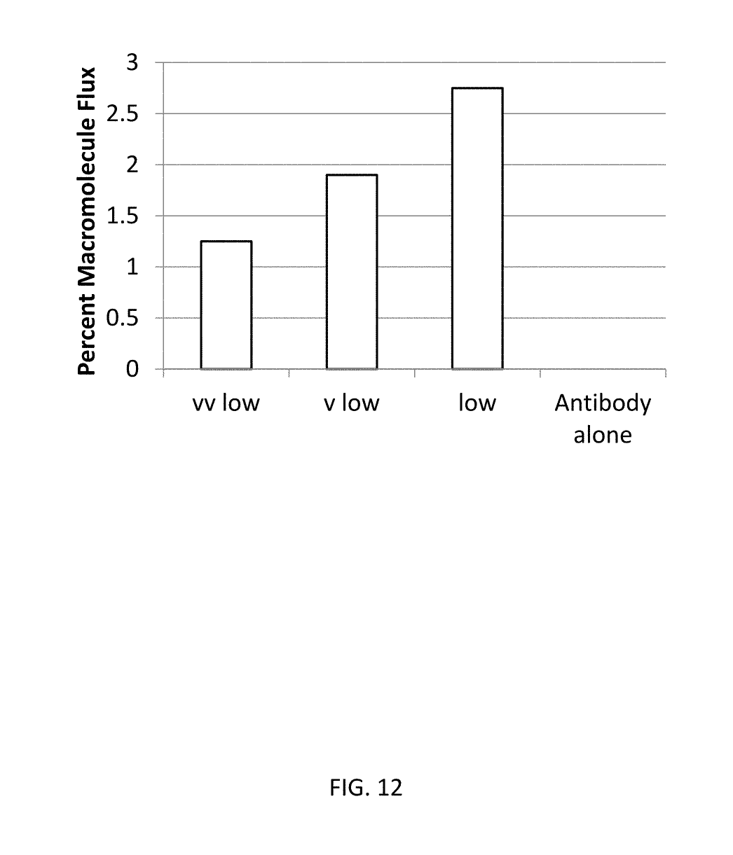

Viable porcine skin was processed to produce mid-dermal grafts (0.045-0.055 units) and the grafts were positioned in transcutaneous flux devices. Flow was maintained at the lowest setting and all receptor fluid was collected for each replicate. Flux was continued for 12-20 hours with samples applied and left on donor surfaces. The skin for each cell (each chamber) was washed and the flow through samples were then employed in an assay of alkaline phosphatase content via absorbance. The results are depicted in FIG. 12.

Antibody alone did not exhibit significant penetration as measured by alkaline phosphatase activity in flow through, while decoy-mediated penetration achieved over 2% penetration of the applied load. A statistically significant increase in penetration (P=0.003) was thus achieved simply by the addition of an decoy molecule. This approach thus affords a high percent penetration which enables development of a topical macromolecule therapeutic. Given that this antibody is 150-160 KD as tagged, delivery of virtually any derivatized antibody or antibody fragment is feasible as is delivery of similar molecules like botulinum toxins and derivatives or chimeras thereof.

### Example 13

### Functional Antioxidant Capacity

Decoys of both hyaluronic acid (HA) and elastin (E6) afford increased penetration of a proprietary mixture of antioxidants from several different formulations. The same antioxidant blend was applied to skin with several different vehicle and decoy combinations as detailed below. Increased resistance to excess functional oxidative stress resulted.

Diffusion Chambers-Viable porcine skin was dermatomed to mid-dermal thickness, then punch biopsies were performed at n=6 per intended condition. A modified 6-block diffusion cell rig was prepared and set for a flow of 0.022 ml/min. The formulations (200 μl each) were applied to the top (donor) surface and massaged. The receptor fluid was collected for 12 hours for each cell for these experiments, then the skin was removed, cleaned, and snap-frozen for future cold homogenization in saline.

Formulations Applied to Porcine
Skin

Saline
Formulation 1
Formulation 1 + 1% HA
Formulation 1 + 0.5% E6
(VGVAPG)
Formulation 2 formulation
Formulation 2 + 1% HA
Formulation 2 + 0.5% E6
Formulation 3
Formulation 3 + 1% HA
Formulation 3 + 0.5% E6
Formulation 3 + 1% HA + 0.5% E6

Invitrogen Amplex Red Kit (Cat#A22188): The Amplex® Red reagent (10-acetyl-3,7-dihydroxyphenoxazine) in the presence of HRP reacts with H₂O₂ in a 1:1 stoichiometry to produce the red-fluorescent oxidation product, resorufin. We employ the kit as a baseline measurement of reactive oxygen species (as the kit was designed) to ensure no aberrant ROS baseline values were present. We then deliberately introduce oxidative stress and watch how each flow-through sample responds. Kit directions were followed for solution prep and reaction setup.

Reactions were incubated at 30° C. for 30 minutes, protected from light and mixed for 5 seconds every 5 minutes (in plate reader). Measure absorbance at 260 nm (reference value to ensure normality) and 560 nm (resorufin) and record values as Baseline (pre-stress). Absorbance was selected instead of fluorescence to allow faster reads post-spike (approximately 1 minute per cycle). For each point, subtract the value derived from average of zero-H₂O₂ control wells (n=2).

Add 20 uL spike of 0.1 mM H₂O₂ stock to each well rapidly then measure absorbance at 260 nm and 560 nm and record values as Stress time zero. Measure dynamic cycles continuously through 5 cycles (approximately 5 minutes) then again at 10 min and 15 min. The multiple reads are to ensure the peak value and linear range can be assessed since resorufin can itself undergo a second oxidation to a non-absorbant/fluorescent state due to the excess H₂O₂ from the spike.

Formulation 1 formulation achieved a mean of 5.15% antioxidant capacity over normal skin controls (saline-treated). Though not statistically significant (p>0.2), the antioxidant capacity of Formulation 1-treated skin was consistently greater than that of saline-treated skin.

All subsequent formulation comparisons were made relative to the Formulation 1 formulation as a reference antioxidant capacity. In this way, the increase in capacity versus current base could be assessed without direct measurement of individual species.

HA Formulations: HA increased the antioxidant capacity of receptor fluid for each base, but there were notable differences from formulation to formulation:

| | Formulation | | |
|---|---|---|---|
| | Native + HA | Sans1 + HA | Sans2 + HA |
| Antioxidant capacity | 200-210% | 414% but rapidly | 316% to 360% |
| versus Native | | declining to appx | |
| | | 100% | |
| Significance | P > 0.2 | *P = 0.019 | *P = 0.029; |
| | | | *P = 0.039 |

Overall, the highest significant antioxidant capacity increases were observed when HA was added to the Formulation 3 base.

E6 afforded consistent increases in antioxidant capacity versus Formulation 1 skin though none achieved p<0.05 (most p<0.08) due to small sample size and lower increases. Since the sans bases were designed around HA behavior rather than E6, there were not significant differences in E6 enhancement from formulation to formulation. Unlike previously observed for other actives in other formulation bases, E6 did not attain as high an increase in antioxidant capacity as observed for HA.

| | Formulation | | |
|---|---|---|---|
| | Native + E6 | Sans1 + E6 | Sans2 + E6 |
| Antioxidant capacity | 162% | 165% | 135% |
| versus Native | | | |

### Example 14

### FTIC-Dextran Confocal Skin Analysis

Real time confocal microscopy imaging was performed on human subjects using a VivaScope® 1500 to visualize penetration of various sized FITC-dextran conjugates up to 150,000 Da across hair bearing skin (dorsal forearm) and non-hair bearing skin (volar forearm). Groups were prepared in saline and consisted of 1% 5,000 Da FITC-dextran or 1% 5,000 Da FITC-dextran plus 1% sodium hyaluronate having average molecular weights of 5,000 Da to 20,000 Da in saline. Similar results were obtained using 0.5% elastin fragments (E6) having molecular weights of 10,000 Da to 20,000 Da in place of HA decoy.

### Example 15

### Summary

It will be appreciated that Examples 1-6, 11, 12 illustrate hyaluronic acid as a decoy molecule exemplary of the decoy molecules contemplated herein. As described above, decoy molecules of collagen and elastin are contemplated, where the molecular weight of the decoy molecule can be selected to tailor the delivery of the agent of interest across the skin. The table below summarizes the effect of the decoy molecule (using the hyaluronic acid as exemplary) on the transdermal delivery of a small molecule compound (e.g, one with a molecular weight of less than about 850 Da), on the transdermal delivery of a macromolecule compound (e.g, a peptide or a protein), on the extent of penetration of the decoy molecule into skin upon topical application, and on the enhancement of water content in skin by the decoy molecule, on a scale using +symbols to reflect extent of the effect. As seen, there is a disconnect between skin penetration of the decoy molecule, hydration of skin due to the decoy molecule and the delivery of the compound into the skin, indicating that the enhanced skin delivery is not due to hydration or presence of the decoy, but to an activity of the decoy molecule in the skin.

| Decoy | Delivery of a | Delivery of | | |
|---|---|---|---|---|
| Molecule - | Small | a Macro- | Hyaluronic | Water Content |
| Hyaluronic | Molecule | molecule | Acid Skin | Enhancement |
| Acid | Compound | Compound | Penetration | in Skin |
| disaccharide | 0 | 0 | +++++ | +++ |
| (400 Da) | | | | |
| degraded | − | − | ++++ | +++ |
| 5000 Da | | | | |
| 3000 Da | +++ | + | ++++ | +++ |
| 5000 Da | + | + | +++ | ++ |
| 10,000 Da | +++ | ++ | ++ | + |
| 20,000 Da | ++++ | +++++ | ++ | + |
| 100,000 Da | ++++ | +++ | +++ | + |
| degraded | | | | |
| 100,000 Da | 0 | 0 | +/− | +/− |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### Example 16

The following compounds will be made and tested for increased flux compared to compositions containing no decoy molecule:

### Collagen and Vitamin C

Compositions containing vitamin C and a decoy molecule of collagen with three molecular weights designated A1, B1, C1 in saline will be prepared. A control formulation comprised of vitamin C in saline will also be tested. The compositions will be placed in Franz diffusion cells with skin separating the compartments of the diffusion cell. The concentration of vitamin C in the receiver side of the diffusion cells will be measured after a fixed time.

### Collagen and Diclofenac

Compositions containing diclofenac and a decoy molecule of collagen with three molecular weights of 5,000 Da, 15,000 Da and 20,000 Da in saline will be prepared. A control formulation comprised of diclofenac in saline will also be tested. The compositions will be placed in Franz diffusion cells with skin separating the compartments of the diffusion cell. The concentration of diclofenac in the receiver side of the diffusion cells will be measured after a fixed time.

### Elastin and Niacinamide

Compositions containing niacinamide and a decoy molecule of elastin with three molecular weights 5,000 Da, 15,000 Da and 20,000 Da in saline will be prepared. A control formulation comprised of niacinamide in saline will also be tested. The compositions will be placed in Franz diffusion cells with skin separating the compartments of the diffusion cell. The concentration of niacinamide in the receiver side of the diffusion cells is measured after a fixed time.

### Elastin and Naproxen

Compositions containing naproxen and a decoy molecule of elastin with three molecular weights 5,000 Da, 15,000 Da, and 20,000 Da in saline will be prepared. A control formulation containing naproxen in saline will also be tested. The compositions will be placed in Franz diffusion cells with skin separating the compartments of the diffusion cell. The concentration of naproxen in the receiver side of the diffusion cells will be measured after a fixed time.

### Topical Administration of Bimatoprost for Hair Growth

Compositions will be prepared containing 0.01% bimatoprost and a 0.5% of a decoy molecule of elastin fragments with one of three molecular weights (650 Da, 800 Da, and 2,000 Da) in saline. Additionally, compositions will be prepared containing 0.01% bimatoprost and 1% of decoy molecule of hyaluronic acid with one of four molecular weights: small (5,000 Da to 10,000 Da), small to mid (10,000 Da to 20,000 Da), low to mid (20,000 Da to 30,000 Da), and mid (30,000 Da to 40,000 Da). Control formulations containing 0.01% bimatoprost alone and saline alone will also be prepared. The compositions will be applied to subjects who have recently completed a cycle of chemotherapy approximately 21 days prior and experienced near total scalp hair loss. Subjects treated with compositions containing either of the decoys are expected to achieve faster rates of hair growth at 1, 2, and 4 weeks relative to comparable controls. Additionally, length, thickness, and density of hair are expected to be greater in subjects treated with compositions containing the decoys.

### Decoy-Enhanced Color Treatment for Hair Shafts

Compositions containing a commercially available hair dye formulations will be spiked with 1% of decoy molecule of hyaluronic acid with low to mid molecular weight (20,000 Da to 30,000 Da) will be prepared and compared to the dye alone. The compositions will be applied to half of scalp hair shafts each (intra-subject control) and will be removed after 30 minutes. The depth of color will be assessed after rinsing, after one week, and after 4 weeks. The hair shafts treated with the composition containing the decoys are expected to demonstrate greater richness, depth, and persistence of color.

1. A composition comprising:
an effective amount of one or more active agents; and
about 0.1 wt. % to about 5.0 wt. % of a extracellular matrix component or a fragment thereof having average molecular weight of about 2,000 daltons to about 60,000 daltons.

2. The composition of claim 1, wherein the extracellular matrix component is selected from the group consisting of hyaluronic acid, collagen, fibronectin, elastin, lectin, and combinations thereof.

3. The composition of claim 2, wherein the collagen is selected from the group consisting of collagen type I, collagen type II, collagen type III, collagen type IV, collagen type V, fibrillary collagen, non-fibrillary collagen, and combinations thereof.

4. The composition of claim 1, wherein the composition comprises about 0.1 wt. % to about 25 wt. % of the active agent.

5. The composition of claim 1, wherein the active agent is selected from the group consisting of analgesic agents, antibacterial agents, antifungal agents, anesthetics, steroids, retinol, gabapentin, pregabalin, minocycline, salicylate, acetyl salicylic acid, cyclosporine, tacrolimus (FK506), hydrocortisone, lidocaine, bimatoprost, botulinum toxin, tadalafil, an antibody, an antibody fragment, and combinations thereof.

6. The composition of claim 1, further comprising one or more pharmaceutical additives selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plastizers, carriers, excipients, and combinations thereof.

7. The composition of claim 1, wherein the composition is formulated as a liquid, cream, ointment, gel, or aerosol.

8. The composition of claim 1, comprising about 0.25 wt. % to about 2.0 wt. % of the extracellular matrix component, and the active agent is selected from the group consisting of botulinum toxin, salicylate, lidocaine, sunblock, retinol, bimatoprost, steroids, and combinations thereof.

9. The composition of claim 1, comprising about 1.0 wt. % to about 5.0 wt. % of the extracellular matrix component, and the active agent is selected from the group consisting of antibiotics, antifungal agents, biologics, antibodies, macromolecule active agents, peptide-based therapeutics, and combinations thereof

10. The composition of claim 1, comprising about 0.1 wt. % to about 2.0 wt. % of the extracellular matrix component, and the active agent is selected from the group consisting of antibiotics, antifungal agents, biologics, antibodies, macromolecule active agents, peptide-based therapeutics, and combinations thereof

11. The composition of claim 1, wherein the extracellular matrix component or a fragment thereof has an average molecular weight of about 2,000 daltons to about 40,000 daltons.

12. The composition of claim 1, wherein the composition is a topical composition.

13. The composition of claim 1, comprising about 0.25 wt. % to about 2.0 wt. % of the extracellular matrix component, and about 0.1 wt. % to about 25 wt. % of the active agent.

14. The composition of claim 1, comprising about 1 wt. % to about 5 wt. % of the extracellular matrix component, and about 0.1 wt. % to about 25 wt. % of the active agent.

15. The composition of claim 1, comprising about 0.1 wt. % to about 2.0 wt. % of the extracellular matrix component, and about 0.1 wt. % to about 25 wt. % of the active agent.

16. The composition of claim 1, wherein the extracellular matrix component is selected from the group consisting of hyaluronic acid, collagen, and combinations thereof.

17. The composition of claim 1, wherein the extracellular matrix component is selected from the group consisting of fibronectin, elastin, lectin, and combinations thereof.

18. The composition of claim 1, wherein the extracellular matrix component is selected from the group consisting of hyaluronic acid, elastin, lectin, and combinations thereof.
